(19) United States
(12) Patent Application Publication
(10) Pub. No.: US 2025/0304575 A1
(43) Pub. Date: Oct. 2, 2025

(54) COMPOUNDS AND PHARMACEUTICAL COMPOSITIONS THEREOF FOR THE TREATMENT OF NEURODEGENERATIVE DISORDERS

(71) Applicant: Galapagos NV, Mechelen (BE)

(72) Inventors: Hans KELGTERMANS, Boortmeerbeek (BE); Jan VAN DER SCHUEREN, Mechelen (BE); Dijana PESIC, Zagreb (HR); Maja Roscic, Zagreb (HR); Mihaela JURKOVIC, Zagreb (HR)

(21) Appl. No.: 18/863,014

(22) PCT Filed: May 3, 2023
(86) PCT No.: PCT/EP2023/061588

§ 371 Date: Nov. 5, 2024

(30) Foreign Application Priority Data
May 5, 2022 (EP) 22171914.9

(51) Int. Cl. C07D 471/04; A61K 31/437; A61P 25/28

(52) U.S. Cl. CPC C07D 471/04; A61K 31/437; A61P 25/28

(57) ABSTRACT

The present invention discloses compounds according to Formula I:

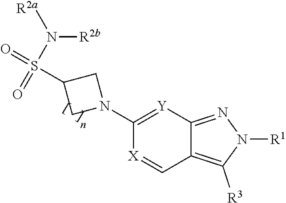

wherein R¹, R^{2a}, R^{2b}, R³, X, Y, and the subscript n are as defined herein.

The present invention relates to compounds, methods for their production, pharmaceutical compositions comprising the same, and methods of treatment using the same, for the prophylaxis and/or treatment of diseases involving neurodegenerative disorders by administering the compound of the invention.

### FIELD OF THE INVENTION

The present invention relates to compounds that may be useful in the prophylaxis and/or treatment of neurodegenerative disorders. The present invention also provides methods for the production of the compound of the invention, pharmaceutical compositions comprising the compound of the invention, methods for the prophylaxis and/or treatment of neurodegenerative disorders by administering the compound of the invention.

### BACKGROUND OF THE INVENTION

Alzheimer's disease (AD) is neurodegenerative disease which can arise sporadically, or with a genetic component, that causes dementia, which is characterized by different clinical symptoms such as a progressive decline in memory, thinking, language, and learning capacity.

It has been established a relation between the accumulation of specific proteins within neurons or in the brain parenchyma, with the development of some particular diseases, known as proteinopathies, including Alzheimer's disease and Parkinson disease (T. Bayer, 2012). This accumulation occurs as a result of a conformational change of an unstructured protein leading to small oligomers that eventually will aggregate into higher order structures.

AD is biologically defined by the presence of 0-amyloid-containing plaques and tau-containing neurofibrillary tangles, and is a common cause of cognitive impairment acquired in midlife and late-life.

The prevalence of cognitive impairment increases exponentially with advancing age and the incidence of dementia increases steeply after 65 years and continues to increase thereafter. The incidence of all-cause dementia in individuals aged 65-70 years is approximately 1 per 100 per year and increases to 4 per 100 per year in those aged 80-90 years.(Knopman et al. 2021). The worldwide prevalence of all types of dementia is expected to increase from 50 million people in 2010 to 113 million by 2050.(Knopman et al. 2021)

The pathology of AD is characterized by lesions including tau-containing neurofibrillary tangles, Aβ-containing plaques, activated glia and/or enlarged endosomes. Additionally, AD also comprises the loss of synaptic homeostasis, neurons or neuronal network integrity.

In AD patients, Aβ-containing extracellular neuritic plaques are found in a widespread distribution throughout the cerebral cortex, and tau-containing neurofibrillary tangles are found initially in the medial temporal lobe and they then spread throughout the isocortical regions of the temporal, parietal and frontal lobes.

Efforts to identify a cure are still struggling to find targets that substantially change the clinical course in persons with AD, and to date no cure has been identified.

### SUMMARY OF THE INVENTION

The present invention relates to compounds that may be useful in the prophylaxis and/or treatment of neurodegenerative disorders. The present invention also provides methods for the production of the compound of the invention, pharmaceutical compositions comprising the compound of the invention, methods for the prophylaxis and/or treatment of neurodegenerative disorders by administering the compound of the invention.

Accordingly, in a first aspect of the invention, the compounds of the invention are provided having a Formula (I):

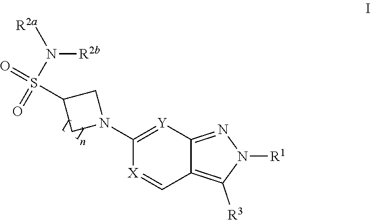

wherein
n is 1, 2 or 3;
one of X and Y is N and the other is CH;
R¹ is phenyl or pyridyl optionally substituted with one or more independently selected R⁴;
R^{2a} and R^{2b} are independently selected from:
   H,
   C₁₋₄ alkyl optionally substituted with one or more independently selected halo, —CN or C₁₋₄ alkoxy, and
   C₃₋₆ cycloalkyl optionally substituted with one or more independently selected halo, —CN or C₁₋₄ alkoxy;
or R^{2a} and R^{2b} together with the N atom may form a 4-7 membered heterocycloalkyl optionally substituted with one or more independently selected halo, —CN or C₁₋₄ alkoxy;
R³ is H, C₁₋₆ alkyl optionally substituted with one or more independently selected halo, or C₃₋₇ monocyclic cycloalkyl optionally substituted with one or more independently selected halo; and each R⁴ is selected from: —OH,
   halo,
   C₁₋₄ alkyl optionally substituted with one or more independently selected halo or C₁₋₄ alkoxy, or
   C₁₋₄ alkoxy optionally substituted with one or more independently selected halo or C₁₋₄ alkoxy.

In a particular aspect, the compounds of the invention are provided for use in the prophylaxis and /or treatment of Alzheimer's disease. In a more particular aspect, the compounds of the invention are provided for use in the treatment of Alzheimer's disease.

Furthermore, the compounds of the inventions might show good exposure in the brain, which might result in good efficacy.

In a further aspect, the present invention provides pharmaceutical compositions comprising a compound of the invention, and a pharmaceutical carrier, excipient or diluent. In a particular aspect, the pharmaceutical composition may additionally comprise further therapeutically active ingredients suitable for use in combination with the compounds of the invention. In a more particular aspect, the further therapeutically active ingredient is an agent for the treatment of neurodegenerative disorders.

Moreover, the compounds of the invention, useful in the pharmaceutical compositions and treatment methods disclosed herein, are pharmaceutically acceptable as prepared and used.

In a further aspect of the invention, this invention provides a method of treating a mammal, in particular humans, afflicted with a condition selected from among those listed herein, and particularly neurodegenerative disorders, which method comprises administering an effective amount of the pharmaceutical composition or compounds of the invention as described herein.

The present invention also provides pharmaceutical compositions comprising a compound of the invention, and a suitable pharmaceutical carrier, excipient or diluent for use in medicine. In a particular aspect, the pharmaceutical composition is for use in the prophylaxis and/or treatment of neurodegenerative disorders.

In additional aspects, this invention provides methods for synthesizing the compounds of the invention, with representative synthetic protocols and pathways disclosed later on herein.

Other objects and advantages will become apparent to those skilled in the art from a consideration of the ensuing detailed description.

### DETAILED DESCRIPTION OF THE INVENTION

### Definitions

The following terms are intended to have the meanings presented therewith below and are useful in understanding the description and intended scope of the present invention.

When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

The articles ‘a’ and ‘an’ may be used herein to refer to one or to more than one (i.e. at least one) of the grammatical objects of the article. By way of example ‘an analogue’ means one analogue or more than one analogue.

‘Alkyl’ means straight or branched aliphatic hydrocarbon having the specified number of carbon atoms. Particular alkyl groups have 1 to 6 carbon atoms or 1 to 4 carbon atoms. Branched means that one or more alkyl groups such as methyl, ethyl or propyl is attached to a linear alkyl chain. Particular alkyl groups are methyl (—CH₃), ethyl (—CH₂—CH₃), n-propyl (—CH₂—CH₂—CH₃), isopropyl (—CH(CH₃)₂), n-butyl (—CH₂—CH₂—CH₂—CH₃), tert-butyl (—C(CH₃)₃), iso-butyl (—CH₂—CH(CH₃)₂), sec-butyl (—CH(CH₃)—CH₂—CH₃), n-pentyl (—CH₂—CH₂—CH₂—CH₂—CH₃), n-hexyl (—CH₂—CH₂—CH₂—CH₂—CH₂—CH₃), and 1,2-dimethylbutyl (—CHCH₃)—C(CH₃)H₂—CH₂—CH₃). Particular alkyl groups have between 1 and 4 carbon atoms.

‘Alkoxy’ refers to the group O-alkyl, where the alkyl group has the number of carbon atoms specified. In particular, the term refers to the group —O—C₁₋₆ alkyl. Particular alkoxy groups are methoxy, ethoxy, n-propoxy, isopropoxy, n-butoxy, tert-butoxy, iso-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

‘Cycloalkyl’ refers to a non-aromatic hydrocarbyl ring structure, monocyclic, fused polycyclic, bridged polycyclic, or spirocyclic, with the number of ring atoms specified. A cycloalkyl may have from 3 to 12 carbon atoms, in particular from 3 to 10, and more particularly from 3 to 7 carbon atoms. Such cycloalkyl groups include, by way of example, single ring structures such as cyclopropyl, cyclobutyl, cyclopentyl, cyclohexyl, and cycloheptyl.

‘Halo’ or ‘halogen’ refer to fluoro (F), chloro (Cl), bromo (Br) and iodo (I). Particular halo groups are either fluoro or chloro.

‘Hetero’ when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g. heteroalkyl, cycloalkyl, e.g. heterocycloalkyl, and the like having from 1 to 4, and particularly from 1 to 3 heteroatoms, more typically 1 or 2 heteroatoms, for example a single heteroatom.

‘Heterocycloalkyl’ means a non-aromatic fully saturated ring structure, monocyclic, fused polycyclic, spirocyclic, or bridged polycyclic, that includes one or more heteroatoms independently selected from O, N and S; and the number of ring atoms specified. The heterocycloalkyl ring structure may have from 4 to 12 ring members, in particular from 4 to 10 ring members and more particularly from 4 to 7 ring members. Each ring may contain up to four heteroatoms typically selected from nitrogen, sulphur and oxygen. Typically, the heterocycloalkyl ring will contain up to 4 heteroatoms, more typically up to 3 heteroatoms, more usually up to 2, for example a single heteroatom. Examples of heterocyclic rings include, but are not limited to azetidinyl, oxetanyl, thietanyl, pyrrolidinyl (e.g. 1-pyrrolidinyl, 2-pyrrolidinyl and 3-pyrrolidinyl), tetrahydrofuranyl (e.g. 1-tetrahydrofuranyl, 2-tetrahydrofuranyl and 3-tetrahydrofuranyl), tetrahydrothiophenyl (e.g. 1-tetrahydrothiophenyl, 2-tetrahydrothiophenyl and 3-tetrahydrothiophenyl), piperidinyl (e.g. 1-piperidinyl, 2-piperidinyl, 3-piperidinyl and 4-piperidinyl), tetrahydropyranyl (e.g. 4-tetrahydropyranyl), tetrahydrothiopyranyl (e.g. 4-tetrahydrothiopyranyl), morpholinyl, thiomorpholinyl, dioxanyl, or piperazinyl.

Particular examples of monocyclic rings are shown in the following illustrative examples:

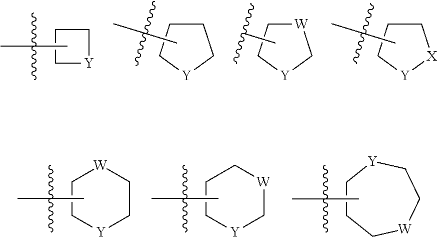

wherein each W and Y is independently selected from —CH₂—, —NH—, —O— and —S—.

‘Substituted’ refers to a group in which one or more hydrogen atoms are each independently replaced with the same or different substituent(s).

‘As used herein, term ‘substituted with one or more’ refers to one to four substituents. In one embodiment it refers to one to three substituents. In further embodiments it refers to one or two substituents.

In a yet further embodiment, it refers to one substituent.

One having ordinary skill in the art of organic synthesis will recognize that the maximum number of heteroatoms in a stable, chemically feasible heterocyclic ring, whether it is aromatic or non-aromatic, is determined by the size of the ring, the degree of unsaturation and the valence of the heteroatoms. In general, a heterocyclic ring may have one to four heteroatoms so long as the heteroaromatic ring is chemically feasible and stable.

‘Pharmaceutically acceptable’ means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

‘Pharmaceutically acceptable salt’ refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g. an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like. The term ‘pharmaceutically acceptable cation’ refers to an acceptable cationic counter-ion of an acidic functional group. Such cations are exemplified by sodium, potassium, calcium, magnesium, ammonium, tetraalkylammonium cations, and the like.

‘Pharmaceutically acceptable vehicle’ refers to a diluent, adjuvant, excipient or carrier with which a compound of the invention is administered.

‘Prodrugs' refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like.

‘Solvate’ refers to forms of the compound that are associated with a solvent, usually by a solvolysis reaction. This physical association includes hydrogen bonding. Conventional solvents include water, EtOH, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. ‘Solvate’ encompasses both solution-phase and isolable solvates. Representative solvates include hydrates, ethanolates and methanolates.

‘Subject’ includes humans. The terms ‘human’, ‘patient’ and ‘subject’ are used interchangeably herein.

‘Effective amount’ means the amount of a compound of the invention that, when administered to a subject for treating a disease, is sufficient to effect such treatment for the disease. The “effective amount” can vary depending on the compound, the disease and its severity, and the age, weight, etc., of the subject to be treated.

‘Preventing’ or ‘prevention’ refers to a reduction in risk of acquiring or developing a disease or disorder (i.e. causing at least one of the clinical symptoms of the disease not to develop in a subject that may be exposed to a disease-causing agent, or predisposed to the disease in advance of disease onset.

The term ‘prophylaxis’ is related to ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

‘Treating’ or ‘treatment’ of any disease or disorder refers, in one embodiment, to ameliorating the disease or disorder (i.e. arresting the disease or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or disorder, either physically, (e.g. stabilization of a discernible symptom), physiologically, (e.g. stabilization of a physical parameter), or both. In a further embodiment, “treating” or “treatment” relates to slowing the progression of the disease.

As used herein the term neurodegenerative diseases refers to proteinopathies, or diseases associated with atrophy of the affected central or peripheral structures of the nervous system. In particular, the term refers to disorders characterized by the accumulation of specific proteins within neurons or in the brain parenchyma, or atrophy of the affected central or peripheral structures of the nervous system. More particularly, the term refers to Alzheimer's disease and other dementias, brain cancer, degenerative nerve diseases, encephalitis, epilepsy, genetic brain disorders, head and brain malformations, hydrocephalus, stroke, Parkinson's disease, multiple sclerosis, amyotrophic lateral sclerosis, Lewy body disease, prion disease, tauopathies, fronto-temporal lobar degeneration, British and Danish dementias, and cerebral autosomal dominant arteriopathy with subcortical infarcts and leukoencephalopathy (CADASIL). Most particularly, the term refers to Alzheimer's disease.

‘Compound(s) of the invention’, and equivalent expressions, are meant to embrace compounds of the Formula(e) as herein described, which expression includes the pharmaceutically acceptable salts, and the solvates, e.g. hydrates, and the solvates of the pharmaceutically acceptable salts where the context so permits. Similarly, reference to intermediates, whether or not they themselves are claimed, is meant to embrace their salts, and solvates, where the context so permits.

When ranges are referred to herein, for example but without limitation, C₁₋₈ alkyl, the citation of a range should be considered a representation of each member of said range.

Other derivatives of the compounds of this invention have activity in both their acid and acid derivative forms, but in the acid sensitive form often offers advantages of solubility, tissue compatibility, or delayed release in the mammalian organism (Bundgaard 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particularly useful prodrugs. In some cases it is desirable to prepare double ester type prodrugs such as (acyloxy)alkyl esters or ((alkoxycarbonyl)oxy)alkylesters. Particular such prodrugs are the C₁₋₈ alkyl, C₂₋₈ alkenyl, C₆₋₁₀ optionally substituted aryl, and (C₆₋₁₀ aryl)-(C₁₋₄ alkyl) esters of the compounds of the invention.

The present disclosure includes all isotopic forms of the compounds of the invention provided herein, whether in a form (i) wherein all atoms of a given atomic number have a mass number (or mixture of mass numbers) which predominates in nature (referred to herein as the “natural isotopic form”) or (ii) wherein one or more atoms are replaced by atoms having the same atomic number, but a mass number different from the mass number of atoms which predominates in nature (referred to herein as an “unnatural variant isotopic form”). It is understood that an atom may naturally exists as a mixture of mass numbers. The term “unnatural variant isotopic form” also includes embodiments in which the proportion of an atom of given atomic number having a mass number found less commonly in nature (referred to herein as an “uncommon isotope”) has been increased relative to that which is naturally occurring e.g. to the level of >20%, >50%, >75%, >90%, >95% or>99% by number of the atoms of that atomic number (the latter embodiment referred to as an “isotopically enriched variant form”). The term “unnatural variant isotopic form” also includes embodiments in which the proportion of an uncommon isotope has been reduced relative to that which is naturally occurring. Isotopic forms may include radioactive forms (i.e. they incorporate radioisotopes) and non-radioactive forms. Radioactive forms will typically be isotopically enriched variant forms.

An unnatural variant isotopic form of a compound may thus contain one or more artificial or uncommon isotopes such as deuterium (²H or D), carbon-11 (¹¹C), carbon-13 (¹³C), carbon-14 (¹⁴C), nitrogen-13 (¹³N), nitrogen-15 (¹⁵N), oxygen-15 (¹¹O), oxygen-17 (¹⁷O), oxygen-18 (¹⁸O), phosphorus-32 (³²P), sulphur-35 (³⁵S), chlorine-36 (³⁶Cl), chlorine-37 (³⁷Cl), fluorine-18 (¹⁸F) iodine-123 (123I), iodine-125 (¹²⁵I) in one or more atoms or may contain an increased proportion of said isotopes as compared with the proportion that predominates in nature in one or more atoms.

Unnatural variant isotopic forms comprising radioisotopes may, for example, be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Unnatural variant isotopic forms which incorporate deuterium i.e ²H or D may afford certain therapeutic advantages resulting from greater metabolic stability, for example, increased in vivo half-life or reduced dosage requirements, and hence may be preferred in some circumstances. Further, unnatural variant isotopic forms may be prepared which incorporate positron emitting isotopes, such as ¹¹C, ¹⁸F, ¹⁵O and ¹³N, and would be useful in Positron Emission Topography (PET) studies for examining substrate receptor occupancy.

It is also to be understood that compounds that have the same molecular formula but differ in the nature or sequence of bonding of their atoms or the arrangement of their atoms in space are termed ‘isomers’. Isomers that differ in the arrangement of their atoms in space are termed ‘stereoisomers’.

Stereoisomers that are not mirror images of one another are termed ‘diastereomers’ and those that are non-superimposable mirror images of each other are termed ‘enantiomers’. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e. as (+) or (−)-isomers respectively).

A chiral compound can exist as either individual enantiomer or as a mixture thereof A mixture containing equal proportions of the enantiomers is called a ‘racemic mixture’.

‘Tautomers' refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro- forms of phenylnitromethane, that are likewise formed by treatment with acid or base.

Tautomeric forms may be relevant to the attainment of the optimal chemical reactivity and biological activity of a compound of interest.

The compounds of the invention may possess one or more asymmetric centers; such compounds can therefore be produced as individual (R)- or (S)- stereoisomers or as mixtures thereof.

Unless indicated otherwise, the description or naming of a particular compound in the specification and claims is intended to include both individual enantiomers and mixtures, racemic or otherwise, thereof.

The methods for the determination of stereochemistry and the separation of stereoisomers are well-known in the art.

It will be appreciated that compounds of the invention may be metabolized to yield biologically active metabolites.

### THE INVENTION

The present invention relates to compounds that may be useful in the prophylaxis and/or treatment of neurodegenerative disorders. The present invention also provides methods for the production of the compound of the invention, pharmaceutical compositions comprising the compound of the invention, methods for the prophylaxis and/or treatment of neurodegenerative disorders by administering the compound of the invention.

Accordingly, in a first aspect of the invention, the compounds of the invention are provided having a Formula (I):

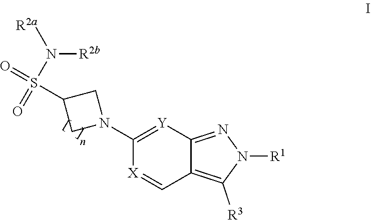

wherein
n is 1, 2 or 3;
one of X and Y is N and the other is CH;
R¹ is phenyl or pyridyl optionally substituted with one or more independently selected R⁴;
R^{2a} and R^{2b} are independently selected from:
   H,
   C₁₋₄ alkyl optionally substituted with one or more independently selected halo, —CN or C₁₋₄ alkoxy, and
   C₃₋₆ cycloalkyl optionally substituted with one or more independently selected halo, —CN or C₁₋₄ alkoxy;
or R^{2a} and R^{2b} together with the N atom may form a 4-7 membered heterocycloalkyl optionally substituted with one or more independently selected halo, —CN or C₁₋₄ alkoxy;
R³ is H, C₁₋₆ alkyl optionally substituted with one or more independently selected halo, or C₃₋₇ monocyclic cycloalkyl optionally substituted with one or more independently selected halo; and
each R⁴ is selected from: —OH,
   halo,
   C₁₋₄ alkyl optionally substituted with one or more independently selected halo or C₁₋₄ alkoxy, or
   C₁₋₄ alkoxy optionally substituted with one or more independently selected halo or C₁₋₄ alkoxy.

In one embodiment, the compound of the invention is according to Formula I, wherein the subscript n is 1, 2 or 3. In a particular embodiment, the subscript n is 2 or 3. In a further particular embodiment, the subscript n is 1.

In one embodiment, the compound of the invention is according to Formula I, wherein R¹ is pyridyl.

In one embodiment, the compound of the invention is according to Formula I, wherein R¹ is pyridyl substituted with one, two or three independently selected R⁴. In a more particular embodiment, R¹ is pyridyl substituted with one R⁴.

In one embodiment, the compound of the invention is according to Formula I, wherein R¹ is phenyl.

In one embodiment, the compound of the invention is according to Formula I, wherein R¹ is phenyl substituted with one or more independently selected R⁴. In a particular embodiment, R¹ is phenyl substituted with one, two or three independently selected R⁴. In a more particular embodiment, R¹ is phenyl substituted with one R⁴.

In one embodiment, the compound of the invention is according to Formula I, wherein R¹ is phenyl substituted with one or more independently selected R⁴, wherein one or more R⁴ is —OH.

In one embodiment, the compound of the invention is according to Formula I, wherein R¹ is phenyl substituted with one or more independently selected R⁴, wherein one or more R⁴ is halo. In a particular embodiment, one or more R⁴ is independently selected from F, Cl and Br. In a more particular embodiment, one or more R⁴ is F.

In one embodiment, the compound of the invention is according to Formula I, wherein R¹ is phenyl substituted with one or more independently selected R⁴, wherein one or more R⁴ is independently selected from C₁₋₄ alkyl optionally substituted with one or more independently selected halo or C₁₋₄ alkoxy. In a particular embodiment, one or more R⁴ is independently selected from —CH₃, —CH₂CH₃, —CH(CH₃)₂, and —C(CH₃)₃, each of which is optionally substituted with one or more independently selected halo or C₁₋₄ alkoxy. In another particular embodiment, one or more R⁴ is independently selected from C₁₋₄ alkyl optionally substituted with one or more independently selected F, Cl, —OCH₃, —OCH₂CH₃. In a more particular embodiment, one or more R⁴ is independently selected from —CH₃, —CH₂CH₃, —CH(CH₃)₂, and -C(CH₃)₃, each of which is optionally substituted with one or more independently selected F, Cl, —OCH₃, or —OCH₂CH₃. In a more particular embodiment, one or more R⁴ is independently selected from —CH₃, and —CF₃.

In one embodiment, the compound of the invention is according to Formula I, wherein R¹ is phenyl substituted with one or more independently selected R⁴, wherein one or more R⁴ is independently selected from C₁₋₄ alkoxy optionally substituted with one or more independently selected halo or C₁₋₄ alkoxy. In a particular embodiment, one or more R⁴ is independently selected from —OCH₃, —OCH₂CH₃, —OCH(CH₃)₂, and —OC(CH₃)₃, each of which is optionally substituted with one or more independently selected halo or C₁. 4 alkoxy. In another particular embodiment, one or more R⁴ is independently selected from C₁₋₄ alkoxy optionally substituted with one or more independently selected F, Cl, —OCH₃, —OCH₂CH₃. In a more particular embodiment, one or more R⁴ is independently selected from —OCH₃, —OCH₂CH₃, —OCH(CH₃)₂, and —OC(CH₃)₃, each of which is optionally substituted with one or more independently selected F, Cl, —OCH₃, or —OCH₂CH₃. In a more particular embodiment, one or more R⁴ is independently selected from —OCH₃, —OCH₂CH₂OCH₃, and —OCF₃.

In one embodiment, the compound of the invention is according to Formula I, wherein R¹ is phenyl substituted with one or more independently selected —OH, F, —CH₃, —CF₃, —OCH₃, —OCH₂CH₂OCH₃, and —OCF₃. In a particular embodiment, R¹ is phenyl substituted with one, or two independently selected —OH, F, —CH₃, —CF₃, —OCH₃, —OCH₂CH₂OCH₃, and —OCF₃.

In one embodiment, the compound of the invention is according to any of Formulae IIa, IIb, IIc or IId:

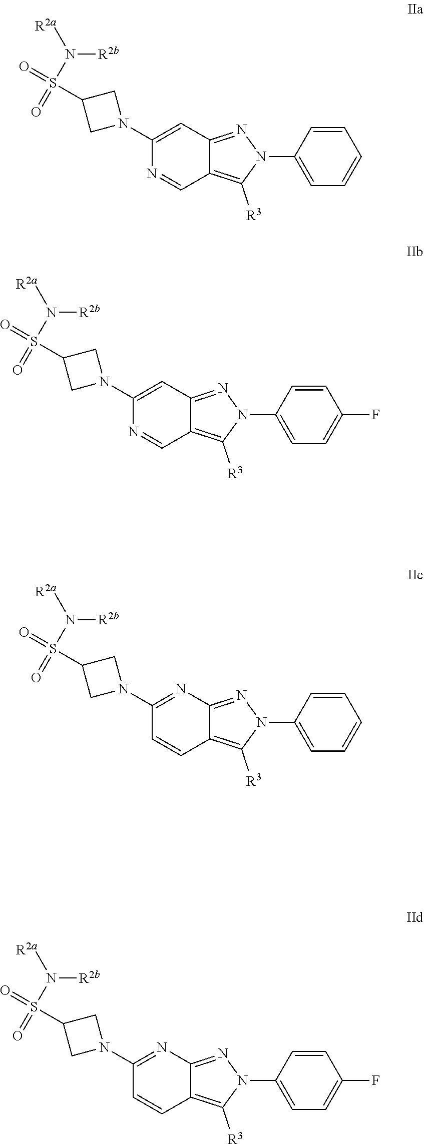

wherein R^{2a}, R^{2b}, and R³ are as described above.

In one embodiment, the compound of the invention is according to any one of Formulae I-IId, wherein R³ is H.

In one embodiment, the compound of the invention is according to any one of Formulae I-IId, wherein R³ is C₁₋₆ alkyl. In a particular embodiment, R³ is —CH₃, —CH₂CH₃, or —CH₂CH(CH₃)₂. In a more particular embodiment, R³ is —CH₃ or —CH₂CH(CH₃)₂. In a most particular embodiment, R³ is —CH₃.

In one embodiment, the compound of the invention is according to any one of Formulae I-IId, wherein R³ is C₁₋₆ alkyl substituted with one or more independently selected halo. In a particular embodiment, R³ is —CH₃, —CH₂CH₃, or —CH₂CH(CH₃)₂, each of which substituted with one or more independently selected halo. In a more particular embodiment, R³ is —CH₃, —CH₂CH₃, or —CH₂CH(CH₃)₂, each of which substituted with one, two or three independently selected halo. In a further particular embodiment, R³ is —CH₃, —CH₂CH₃, or —CH₂CH(CH₃)₂, each of which substituted with one, two or three independently selected F, Cl or Br. In an even more particular embodiment, R³ is —CH₃, —CH₂CH₃, or —CH₂CH(CH₃)₂, each of which substituted with one, two or three independently selected F or Cl. In a most particular embodiment, R³ is —CH₃, —CH₂CH₃, or —CH₂CH(CH₃)₂, each of which substituted with one, two or three F. In a further most particular embodiment, R³ is —CF₃.

In one embodiment, the compound of the invention is according to any one of Formulae I-IId, wherein R³ is C₃₋₇ monocyclic cycloalkyl substituted with one or more independently selected halo. In a particular embodiment, R³ is cyclopropyl, cyclobutyl, or cyclopentyl, each of which substituted with one, two or three independently selected halo. In a more particular embodiment, R³ is cyclopropyl, cyclobutyl, or cyclopentyl, each of which substituted with one, two or three independently selected F, Cl or Br. In a further particular embodiment, R³ is cyclopropyl, cyclobutyl, or cyclopentyl, each of which substituted with one, two or three independently selected F or Cl. In an even more particular embodiment, R³ is cyclopropyl, cyclobutyl, or cyclopentyl, each of which substituted with one, two or three F.

In one embodiment, the compound of the invention is according to any one of Formulae I-IId, wherein R³ is C₃₋₇ monocyclic cycloalkyl. In a particular embodiment, R³ is cyclopropyl, cyclobutyl, or cyclopentyl. In a particular embodiment, R³ is cyclopropyl.

In one embodiment, the compound of the invention is according to any of Formulae IIIa, IIIb, IIIc, IIId, IIIe or IIIf:

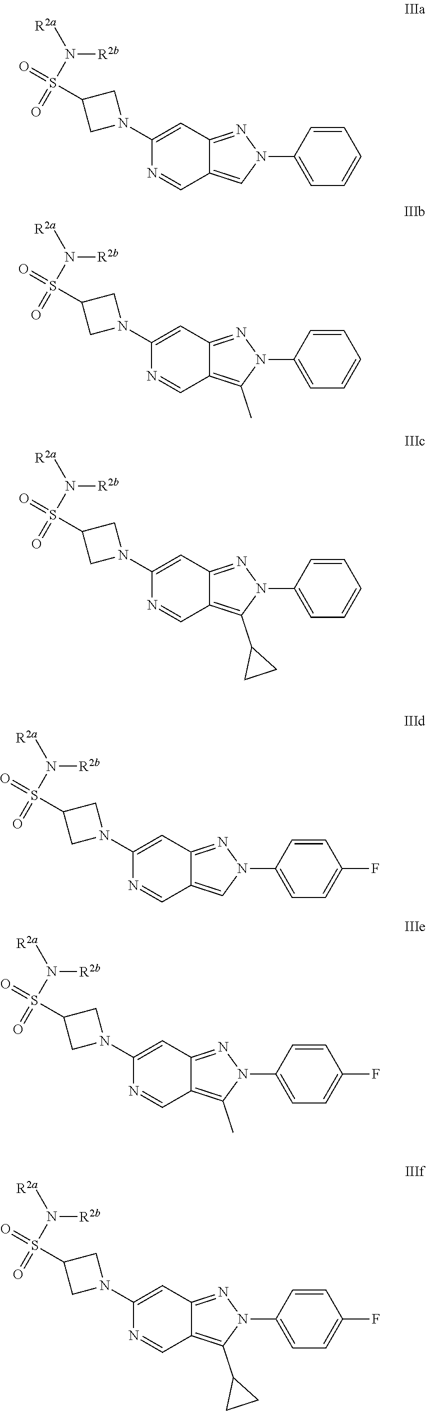

wherein R^{2a} and R^{2b} are as described above.

In one embodiment, the compound of the invention is according to any one of Formulae I-IIIf, wherein each R^{2a} and R^{2b} are both H.

In one embodiment, the compound of the invention is according to any one of Formulae I-IIIf, wherein each R^{2a} and R^{2b} are independently selected from H, C₁₋₄ alkyl, and C₃₋₆ cycloalkyl. In a particular embodiment, each R^{2a} and R^{2b} are independently selected from H, —CH₃, —CH₂CH₃, —CH₂CH(CH₃)₂, cyclopropyl, cyclobutyl, and cyclopentyl. In another particular embodiment, R^{2a} is H and R^{2b} is selected from H, —CH₃, —CH₂CH₃, —CH₂CH(CH₃)₂, cyclopropyl, cyclobutyl, and cyclopentyl. In a particular embodiment, each R^{2a} and R^{2b} is independently selected from —CH₃, —CH₂CH₃, —CH₂CH(CH₃)₂, cyclopropyl, cyclobutyl, and cyclopentyl. In a most particular embodiment, R^{2a} and R^{2b} are both —CH₃.

In one embodiment, the compound of the invention is according to any one of Formulae I-IIIf, wherein R^{2a} and R^{2b} together with the N atom may form a 4-7 membered heterocycloalkyl. In a particular embodiment, R^{2a} and R^{2b} together with the N atom may form a azetidinyl, pyrrolidinyl, or piperidinyl ring.

In a more particular embodiment, R^{2a} and R^{2b} together with the N atom are

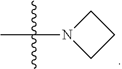 [SMILES: *N1CCC1]

In one embodiment, the compound of the invention is according to Formula I is selected from:
N,N-dimethyl-1-(2-phenyl-2H-pyrazolo[4,3-c]pyridin-6-yl)azetidine-3-sulfonamide,
N,N-dimethyl-1-(2-phenyl-2H-pyrazolo[4,3-c]pyridin-6-yl) pyrrolidine-3-sulfonamide,
N,N-dimethyl-1-(2-phenyl-2H-pyrazolo[4,3-c]pyridin-6-yl)piperidine-3-sulfonamide,
N-isopropyl-N-methyl-1-(2-phenyl-2H-pyrazolo[4,3-c]pyridin-6-yl)azetidine-3-sulfonamide,
N-cyclopropyl-N-methyl-1-(2-phenyl-2H-pyrazolo[4,3-c]pyridin-6-yl)azetidine-3-sulfonamide,
1-(2-phenyl-2H-pyrazolo[4,3-c]pyridin-6-yl)azetidine-3-sulfonamide,
1-(2-(4-methoxyphenyl)-2H-pyrazolo[4,3-c]pyridin-6-yl)-N,N-dimethylazetidine-3-sulfonamide,
1-(2-(4-methoxyphenyl)-2H-pyrazolo[4,3-c]pyridin-6-yl)-N,N-dimethylpyrrolidine-3-sulfonamide,
1-(2-(4-fluorophenyl)-2H-pyrazolo[4,3-c]pyridin-6-yl)-N,N-dimethylazetidine-3-sulfonamide,
1-(2-(4-fluorophenyl)-2H-pyrazolo[4,3-c]pyridin-6-yl)-N,N-dimethylpyrrolidine-3-sulfonamide,
1-(2-(3-methoxyphenyl)-2H-pyrazolo[4,3-c]pyridin-6-yl)-N,N-dimethylazetidine-3-sulfonamide,
1-(2-(3-methoxyphenyl)-2H-pyrazolo[4,3-c]pyridin-6-yl)-N,N-dimethylpyrrolidine-3-sulfonamide,
N,N-dimethyl-1-(2-(4-(trifluoromethoxy)phenyl)-2H-pyrazolo[4,3-c]pyridin-6-yl)azetidine-3-sulfonamide,
1-(2-(2-fluorophenyl)-2H-pyrazolo[4,3-c]pyridin-6-yl)-N,N-dimethylazetidine-3-sulfonamide,
1-(2-(2-fluorophenyl)-2H-pyrazolo[4,3-c]pyridin-6-yl)-N,N-dimethylpyrrolidine-3-sulfonamide,
N,N-dimethyl-1-(2-(4-(trifluoromethoxy)phenyl)-2H-pyrazolo[4,3-c]pyridin-6-yl)pyrrolidine-3-sulfonamide,
1-(2-(2-methoxyphenyl)-2H-pyrazolo[4,3-c]pyridin-6-yl)-N,N-dimethylazetidine-3-sulfonamide,
N,N-dimethyl-1-(2-(o-tolyl)-2H-pyrazolo[4,3-c]pyridin-6-yl)azetidine-3-sulfonamide,
1-(2-(2-methoxy-6-methylphenyl)-2H-pyrazolo[4,3-c]pyridin-6-yl)-N,N-dimethylazetidine-3-sulfonamide,
1-(2-(2,6-dimethylphenyl)-2H-pyrazolo[4,3-c]pyridin-6-yl)-N,N-dimethylazetidine-3-sulfonamide,
N,N-dimethyl-1-(3-methyl-2-phenyl-2H-pyrazolo[4,3-c]pyridin-6-yl)azetidine-3-sulfonamide,
N,N-dimethyl-1-(2-(4-(trifluoromethyl)phenyl)-2H-pyrazolo[4,3-c]pyridin-6-yl)azetidine-3-sulfonamide,
N,N-dimethyl-1-(2-(p-tolyl)-2H-pyrazolo[4,3-c]pyridin-6-yl)azetidine-3-sulfonamide,
1-(2-(4-hydroxyphenyl)-2H-pyrazolo[4,3-c]pyridin-6-yl)-N,N-dimethylazetidine-3-sulfonamide,
1-(2-(4-(2-methoxyethoxy)phenyl)-2H-pyrazolo[4,3-c]pyridin-6-yl)-N,N-dimethylazetidine-3-sulfonamide,
1-(2-(4-fluorophenyl)-3-methyl-2H-pyrazolo[4,3-c]pyridin-6-yl)-N,N-dimethylazetidine-3-sulfonamide,
1-(2-(4-fluorophenyl)-2H-pyrazolo[4,3-c]pyridin-6-yl)azetidine-3-sulfonamide,
1-(2-(2-(2-methoxyethoxy)phenyl)-2H-pyrazolo[4,3-c]pyridin-6-yl)-N,N-dimethylazetidine-3-sulfonamide,
1-(2-(3-(2-methoxyethoxy)phenyl)-2H-pyrazolo[4,3-c]pyridin-6-yl)-N,N-dimethylazetidine-3-sulfonamide,
1-(2-(4-fluorophenyl)-2H-pyrazolo[4,3-c]pyridin-6-yl)-N-methylazetidine-3-sulfonamide,
N-methyl-1-(2-phenyl-2H-pyrazolo[4,3-c]pyridin-6-yl)azetidine-3-sulfonamide,
N-methyl-1-(3-methyl-2-phenyl-2H-pyrazolo[4,3-c]pyridin-6-yl)azetidine-3-sulfonamide,
1-(3-methyl-2-phenyl-2H-pyrazolo[4,3-c]pyridin-6-yl)azetidine-3-sulfonamide,
1-(3-cyclopropyl-2-phenyl-2H-pyrazolo[4,3-c]pyridin-6-yl)-N,N-dimethylazetidine-3-sulfonamide,
1-(2-(4-fluorophenyl)-3-methyl-2H-pyrazolo[4,3-c]pyridin-6-yl)-N-methylazetidine-3-sulfonamide,
1-(2-(4-fluorophenyl)-3-methyl-2H-pyrazolo[4,3-c]pyridin-6-yl)azetidine-3-sulfonamide,
1-(3-cyclopropyl-2-(4-fluorophenyl)-2H-pyrazolo[4,3-c]pyridin-6-yl)-N,N-dimethylazetidine-3-sulfonamide,
1-(2-(4-fluorophenyl)-3-isopropyl-2H-pyrazolo[4,3-c]pyridin-6-yl)-N,N-dimethylazetidine-3-sulfonamide,
6-(3-(azetidin-1-ylsulfonyl)azetidin-1-yl)-2-phenyl-2H-pyrazolo[4,3-c]pyridine,
6-(3-(azetidin-1-ylsulfonyl)azetidin-1-yl)-2-(4-fluorophenyl)-2H-pyrazolo[4,3-c]pyridine,
N,N-dimethyl-1-(2-phenyl-2H-pyrazolo[3,4-b]pyridin-6-yl)azetidine-3-sulfonamide,
1-(2-(4-fluorophenyl)-2H-pyrazolo[3,4-b]pyridin-6-yl)-N,N-dimethylazetidine-3-sulfonamide,
N-methyl-1-(2-phenyl-2H-pyrazolo[3,4-b]pyridin-6-yl)azetidine-3-sulfonamide, and
1-(2-phenyl-2H-pyrazolo[3,4-b]pyridin-6-yl)azetidine-3-sulfonamide.

In one embodiment, the compounds of the invention are provided in a natural isotopic form.

In one embodiment, the compounds of the invention are provided in an unnatural variant isotopic form. In a specific embodiment, the unnatural variant isotopic form is a form in which deuterium (i.e. ²H or D) is incorporated where hydrogen is specified in the chemical structure in one or more atoms of a compound of the invention. In one embodiment, the atoms of the compounds of the invention are in an isotopic form which is not radioactive. In one embodiment, one or more atoms of the compounds of the invention are in an isotopic form which is radioactive. Suitably radioactive isotopes are stable isotopes.

Suitably the unnatural variant isotopic form is a pharmaceutically acceptable form.

In one embodiment, a compound of the invention is provided whereby a single atom of the compound exists in an unnatural variant isotopic form. In another embodiment, a compound of the invention is provided whereby two or more atoms exist in an unnatural variant isotopic form.

Unnatural isotopic variant forms can generally be prepared by conventional techniques known to those skilled in the art or by processes described herein e.g. processes analogous to those described in the accompanying Examples for preparing natural isotopic forms. Thus, unnatural isotopic variant forms could be prepared by using appropriate isotopically variant (or labelled) reagents in place of the normal reagents employed in the illustrative example as examples.

In one aspect a compound of the invention according to any one of the embodiments herein described is present as the free base.

In one aspect a compound of the invention according to any one of the embodiments herein described is a pharmaceutically acceptable salt.

In one aspect a compound of the invention according to any one of the embodiments herein described is a solvate of the compound.

In one aspect a compound of the invention according to any one of the embodiments herein described is a solvate of a pharmaceutically acceptable salt of a compound.

While specified groups for each embodiment have generally been listed above separately, a compound of the invention includes one in which several or each embodiment in the above Formula, as well as other formulae presented herein, is selected from one or more of particular members or groups designated respectively, for each variable. Therefore, this invention is intended to include all combinations of such embodiments within its scope.

While specified groups for each embodiment have generally been listed above separately, a compound of the invention may be one for which one or more variables (for example, R groups) is selected from one or more embodiments according to any of the Formula(e) listed above. Therefore, the present invention is intended to include all combinations of variables from any of the disclosed embodiments within its scope.

Alternatively, the exclusion of one or more of the specified variables from a group or an embodiment, or combinations thereof is also contemplated by the present invention.

In certain aspects, the present invention provides prodrugs and derivatives of the compounds according to the formulae above. Prodrugs are derivatives of the compounds of the invention, which have metabolically cleavable groups and become by solvolysis or under physiological conditions the compounds of the invention, which are pharmaceutically active, in vivo. Such examples include, but are not limited to, choline ester derivatives and the like, N-alkylmorpholine esters and the like.

Other derivatives of the compounds of this invention have activity in both their acid and acid derivative forms, but the acid sensitive form often offers advantages of solubility, tissue compatibility, or delayed release in the mammalian organism (Bundgard, H, 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Particularly useful are the C₁ to C₈ alkyl, C₂-C₈ alkenyl, aryl, C₇-C₁₂ substituted aryl, and C₇-C₁₂ arylalkyl esters of the compounds of the invention.

### Pharmaceutical Compositions

When employed as a pharmaceutical, a compound of the invention is typically administered in the form of a pharmaceutical composition. Such compositions can be prepared in a manner well known in the pharmaceutical art and comprise at least one active compound of the invention according to Formula I. Generally, a compound of the invention is administered in a pharmaceutically effective amount. The amount of compound of the invention actually administered will typically be determined by a physician, in the light of the relevant circumstances, including the condition to be treated, the chosen route of administration, the actual compound of the invention administered, the age, weight, and response of the individual patient, the severity of the patient's symptoms, and the like.

The pharmaceutical compositions of this invention can be administered by a variety of routes including oral, rectal, transdermal, subcutaneous, intra-articular, intravenous, intramuscular, and intranasal. Depending on the intended route of delivery, a compound of the invention is preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term ‘unit dosage forms’ refers to physically discrete units suitable as unitary dosages for human subjects and other mammals, each unit containing a predetermined quantity of active material calculated to produce the desired therapeutic effect, in association with a suitable pharmaceutical excipient, vehicle or carrier. Typical unit dosage forms include prefilled, premeasured ampules or syringes of the liquid compositions or pills, tablets, capsules or the like in the case of solid compositions. In such compositions, the compound of the invention according to Formula I is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or non-aqueous vehicle with buffers, suspending and dispensing agents, colorants, flavors and the like. Solid forms may include, for example, any of the following ingredients, or compound of the inventions of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint or orange flavoring.

Injectable compositions are typically based upon injectable sterile saline or phosphate-buffered saline or other injectable carriers known in the art. As before, the active compound of the invention according to Formula I in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

A compound of the invention can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

A compound of the invention can also be administered in sustained release forms or from sustained release drug delivery systems. A description of representative sustained release materials can be found in Remington's Pharmaceutical Sciences.

The following formulation examples illustrate representative pharmaceutical compositions that may be prepared in accordance with this invention. The present invention, however, is not limited to the following pharmaceutical compositions.

### Formulation 1—Tablets

A compound of the invention according to Formula I may be admixed as a dry powder with a dry gelatin binder in an approximate 1:2 weight ratio. A minor amount of magnesium stearate may be added as a lubricant. The mixture may be formed into 240-270 mg tablets (80-90 mg of active compound of the invention according to Formula I per tablet) in a tablet press.

### Formulation 2—Capsules

A compound of the invention according to Formula I may be admixed as a dry powder with a starch diluent in an approximate 1:1 weight ratio. The mixture may be filled into 250 mg capsules (125 mg of active compound of the invention according to Formula I per capsule).

### Formulation 3—Liquid

A compound of the invention according to Formula 1(125 mg), may be admixed with sucrose (1.75 g) and xanthan gum (4 mg) and the resultant mixture may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color may be diluted with water and added with stirring. Sufficient water may then be added with stirring. Further sufficient water may be then added to produce a total volume of 5 mL.

### Formulation 4—Tablets

A compound of the invention according to Formula I may be admixed as a dry powder with a dry gelatin binder in an approximate 1:2 weight ratio. A minor amount of magnesium stearate may be added as a lubricant. The mixture may be formed into 450-900 mg tablets (150-300 mg of active compound of the invention according to Formula I) in a tablet press.

### Formulation 5—Injection

A compound of the invention according to Formula I may be dissolved or suspended in a buffered sterile saline injectable aqueous medium to a concentration of approximately 5 mg/mL.

### Formulation 6—Topical

Stearyl alcohol (250 g) and a white petrolatum (250 g) may be melted at about 75° C. and then a mixture of A compound of the invention according to Formula I (50 g) methylparaben (0.25 g), propylparaben (0.15 g), sodium lauryl sulfate (10 g), and propylene glycol (120 g) dissolved in water (about 370 g) may be added and the resulting mixture may be stirred until it congeals.

### Methods of Treatment

In one embodiment, the present invention provides compounds of the invention, or pharmaceutical compositions comprising a compound of the invention, for use in medicine.

In a particular embodiment, the present invention provides compounds of the invention or pharmaceutical compositions comprising a compound of the invention, for use in the prophylaxis and/or treatment of neurodegenerative diseases. In particular, the term neurodegenerative diseases refers to proteinopathies, or atrophy of the affected central or peripheral structures of the nervous system. More particularly, the term neurodegenerative diseases refers to Alzheimer's disease and other dementias, brain cancer, degenerative nerve diseases, encephalitis, epilepsy, genetic brain disorders, head and brain malformations, hydrocephalus, stroke, Parkinson's disease, multiple sclerosis, amyotrophic lateral sclerosis, Lewy body disease, prion disease, tauopathies, fronto-temporal lobar degeneration, British and Danish dementias, and cerebral autosomal dominant arteriopathy with subcortical infarcts and leukoencephalopathy (CADASIL). Most particularly, the term neurodegenerative diseases refers to Alzheimer's disease.

In another embodiment, the present invention provides the use of compounds of the invention or pharmaceutical compositions comprising a compound of the invention in the manufacture of a medicament for the prophylaxis and/or treatment of neurodegenerative diseases. In particular, the term neurodegenerative diseases refers to proteinopathies, or atrophy of the affected central or peripheral structures of the nervous system. More particularly, the term neurodegenerative diseases refers to Alzheimer's disease and other dementias, brain cancer, degenerative nerve diseases, encephalitis, epilepsy, genetic brain disorders, head and brain malformations, hydrocephalus, stroke, Parkinson's disease, multiple sclerosis, amyotrophic lateral sclerosis, Lewy body disease, prion disease, tauopathies, fronto-temporal lobar degeneration, British and Danish dementias, and cerebral autosomal dominant arteriopathy with subcortical infarcts and leukoencephalopathy (CADASIL). Most particularly, the term neurodegenerative diseases refers to Alzheimer's disease.

In additional method of treatment aspects, this invention provides methods of prophylaxis and/or treatment of a mammal afflicted with neurodegenerative diseases, which methods comprise the administration of an effective amount of a compound of the invention or one or more of the pharmaceutical compositions herein described for the treatment or prophylaxis of said condition. In particular, the term neurodegenerative diseases refers to proteinopathies, or atrophy of the affected central or peripheral structures of the nervous system. More particularly, the term neurodegenerative diseases refers to Alzheimer's disease and other dementias, brain cancer, degenerative nerve diseases, encephalitis, epilepsy, genetic brain disorders, head and brain malformations, hydrocephalus, stroke, Parkinson's disease, multiple sclerosis, amyotrophic lateral sclerosis, Lewy body disease, prion disease, tauopathies, fronto-temporal lobar degeneration, British and Danish dementias, and cerebral autosomal dominant arteriopathy with subcortical infarcts and leukoencephalopathy (CADASIL). Most particularly, the term neurodegenerative diseases refers to Alzheimer's disease.

In one embodiment, the present invention provides pharmaceutical compositions comprising a compound of the invention, and another therapeutic agent. In a particular embodiment, the other therapeutic agent is a neurodegenerative diseases treatment agent. In particular, the term neurodegenerative diseases refers to proteinopathies, or atrophy of the affected central or peripheral structures of the nervous system. More particularly, the term neurodegenerative diseases refers to Alzheimer's disease and other dementias, brain cancer, degenerative nerve diseases, encephalitis, epilepsy, genetic brain disorders, head and brain malformations, hydrocephalus, stroke, Parkinson's disease, multiple sclerosis, amyotrophic lateral sclerosis, Lewy body disease, prion disease, tauopathies, fronto-temporal lobar degeneration, British and Danish dementias, and cerebral autosomal dominant arteriopathy with subcortical infarcts and leukoencephalopathy (CADASIL). Most particularly, the term neurodegenerative diseases refers to Alzheimer's disease. In another embodiment, the another therapeutic agent is selected from aducanumab, galantamine, rivastigmine, donepezil and memantine.

Injection dose levels range from about 0.1 mg/kg/h to at least 10 mg/kg/h, all for from about 1 to about 120 h and especially 24 to 96 h. A preloading bolus of from about 0.1 mg/kg to about 10 mg/kg or more may also be administered to achieve adequate steady state levels. The maximum total dose is not expected to exceed about 1 g/day for a 40 to 80 kg human patient.

For the prophylaxis and/or treatment of long-term conditions, such as degenerative conditions, the regimen for treatment usually stretches over many months or years so oral dosing is preferred for patient convenience and tolerance. With oral dosing, one to four (1-4) regular doses daily, especially one to three (1-3) regular doses daily, typically one to two (1-2) regular doses daily, and most typically one (1) regular dose daily are representative regimens. Alternatively for long lasting effect drugs, with oral dosing, once every other week, once weekly, and once a day are representative regimens. In particular, dosage regimen can be every 1-14 days, more particularly 1-10 days, even more particularly 1-7 days, and most particularly 1-3 days.

Using these dosing patterns, each dose provides from about ito about 1000 mg of a compound of the invention, with particular doses each providing from about 10 to about 500 mg and especially about 30 to about 250 mg

Transdermal doses are generally selected to provide similar or lower blood levels than are achieved using injection doses.

When used to prevent the onset of a condition, a compound of the invention will be administered to a patient at risk for developing the condition, typically on the advice and under the supervision of a physician, at the dosage levels described above. Patients at risk for developing a particular condition generally include those that have a family history of the condition, or those who have been identified by genetic testing or screening to be particularly susceptible to developing the condition.

A compound of the invention can be administered as the sole active agent or it can be administered in combination with other therapeutic agents, including other compound of the inventions that demonstrate the same or a similar therapeutic activity and that are determined to be safe and efficacious for such combined administration. In a specific embodiment, co-administration of two (or more) agents allows for significantly lower doses of each to be used, thereby reducing the side effects seen.

In one embodiment, a compound of the invention or a pharmaceutical composition comprising a compound of the invention is administered as a medicament. In a specific embodiment, said pharmaceutical composition additionally comprises a further active ingredient.

In one embodiment, a compound of the invention is co-administered with another therapeutic agent for the treatment and/or prophylaxis of neurodegenerative diseases, particular agents include but are not limited to: amantadine, apomorphine, aducanumab, baclofen, carbidopa, carbidopa, dantrolene, donepezil, entacapone, galantamine, levodopa, memantine, pramipexole, rasagiline, riluzole, rivastigmine, ropinirole, selegiline, tacrine, tetrabenazine, tizanidine, and tolcapone.

In one embodiment, a compound of the invention is co-administered with another therapeutic agent for the treatment and/or prophylaxis of proliferative disorders, particular agents include but are not limited to: methotrexate, leukovorin, adriamycin, prednisone, bleomycin, cyclophosphamide, 5-fluorouracil, paclitaxel, docetaxel, vincristine, vinblastine, vinorelbine, doxorubicin, tamoxifen, toremifene, megestrol acetate, anastrozole, goserelin, anti-HER2 monoclonal antibody (e.g. Herceptin™), capecitabine, raloxifene hydrochloride, EGFR inhibitors (e.g. Iressa®, Tarceva™, Erbitux™), VEGF inhibitors (e.g. Avastin™), proteasome inhibitors (e.g. Velcade™), Glivec® and hsp90 inhibitors (e.g. 17-AAG). Additionally, the compound of the invention according to Formula I may be administered in combination with other therapies including, but not limited to, radiotherapy or surgery. In a specific embodiment the proliferative disorder is selected from cancer, myeloproliferative disease or leukaemia.

In one embodiment, a compound of the invention is co-administered with another therapeutic agent for the treatment and/or prophylaxis of autoimmune diseases, particular agents include but are not limited to: glucocorticoids, cytostatic agents (e.g. purine analogs), alkylating agents, (e.g nitrogen mustards (cyclophosphamide), nitrosoureas, platinum compound of the inventions, and others), antimetabolites (e.g. methotrexate, azathioprine and mercaptopurine), cytotoxic antibiotics (e.g. dactinomycin anthracyclines, mitomycin C, bleomycin, and mithramycin), antibodies (e.g. anti-CD20, anti-CD25 or anti-CD3 (OTK3) monoclonal antibodies, Atgam® and Thymoglobuline@), cyclosporin, tacrolimus, rapamycin (sirolimus), interferons (e.g. IFN-0), TNF binding proteins (e.g. infliximab, etanercept, or adalimumab), mycophenolate, fingolimod and myriocin..

By co-administration is included any means of delivering two or more therapeutic agents to the patient as part of the same treatment regime, as will be apparent to the skilled person. Whilst the two or more agents may be administered simultaneously in a single formulation, i.e. as a single pharmaceutical composition, this is not essential. The agents may be administered in different formulations and at different times.

### CHEMICAL SYNTHETIC PROCEDURES

### General

The compound of the invention can be prepared from readily available starting materials using the following general methods and procedures. It will be appreciated that where typical or preferred process conditions (i.e. reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art (“Greene's Protective Groups in Organic Synthesis, 4th Edition Wiley” 2006).

The following methods are presented with details as to the preparation of a compound of the invention as defined hereinabove and the comparative examples. A compound of the invention may be prepared from known or commercially available starting materials and reagents by one skilled in the art of organic synthesis.

All reagents were of commercial grade and were used as received without further purification, unless otherwise stated. Commercially available anhydrous solvents were used for reactions conducted under inert atmosphere. Reagent grade solvents were used in all other cases, unless otherwise specified. Column chromatography was performed on silica gel 60 (35-70 μm). Thin layer chromatography was carried out using pre-coated silica gel F-254 plates (thickness 0.25 mm). ¹H NMR spectra were recorded on a Bruker DPX 400 NMR spectrometer (400 MHz or a Bruker Advance 300 NMR spectrometer (300 MHz). Chemical shifts (6) for 1H NMR spectra are reported in parts per million (ppm) relative to tetramethylsilane (6 0.00) or the appropriate residual solvent peak, i.e. CHCl₃ (6 7.27), as internal reference. Multiplicities are given as singlet (s), doublet (d), triplet (t), quartet (q), quintuplet (quin), multiplet (in) and broad (br). Electrospray MS spectra were obtained on a Waters platform LC/MS spectrometer or with Waters Acquity H-Class UPLC coupled to a Waters Mass detector 3100 spectrometer. Columns used: Waters Acquity UPLC BEH C18 1.7 μm, 2.1 mm ID ×50 mm L, Waters Acquity UPLC BEH C18 1.7 μm, 2.1 mm ID×30 mm L, or Waters Xterra MS 5 μm C18, 100×4.6 mm. The methods are using either MeCN/H₂O gradients (H₂O contains either 0.1% TFA or 0.1% NH₃) or MeOH/H₂O gradients (H₂O contains 0.0500 TFA). Microwave heating was performed with a Biotage Initiator.

TABLE I
List of abbreviations used in the experimental section

| Abbreviation | Definition | Abbreviation | Definition |
|---|---|---|---|
| μL | microliter | Int | intermediate |
| ACN | acetonitrile | iPrOH | isopropanol |
| AcOH | acetic acid | m | multiplet |
| aq. | aqueous | MeCN | acetonitrile |
| atm | atmosphere | MeOH | methanol |
| Boc | tert-butyloxy-carbonyl | mg | milligram |
| br | broad signal | MHz | megahertz |
| BSA | bovine serum albumin | min | minute |
| calc | calculated | mL | milliliter |
| Cpd | compound | mmol | millimole |
| d | doublet | mol | mole |
| δ | chemical shift | MOPS | 3-(N-morpholino)propanesulfonic acid |
| DCM | dichloromethane | MS | mass spectrometry |
| DMEM | Dulbecco's Modified Eagle's Medium | MW | molecular weight |
| DMF | dimethylformamide | N | normality |
| DMSO | dimethylsulfoxide | ND | Not determined |
| DPBS | Dulbecco's Phosphate Buffered Saline | NMR | nuclear magnetic resonance |
| EDTA | ethylenediaminetetraacetic acid | Obs | observed |
| eq. | equivalent | PBS | phosphate-buffered saline |
| ES− | electrospray negative | PBST | phosphate-buffered saline with Tween 20 |
| ES+ | electrospray positive | Pd/C | palladium on carbon |
| EtOAc | ethyl acetate | PMB | p-methoxybenzyl |
| EtOH | ethanol | ppm | part-per-million |
| FBS | fetal bovine serum | q | quartet |
| g | gram | r.t. | room temperature |
| KO^{t}Bu | potassium tert-butoxide | s | singlet |
| h | hour | sat. | saturated |
| HPLC | high performance liquid chromatography | SCX | strong cation exchange |
| Hz | hertz | SFC | supercritical fluid chromatography |
| MTBE | methyl tert-butyl ether | XPhos | dicyclohexylphosphino-2,4,6-tri-i- |
| TEA | triethylamine | Pd G3 | propyl-1,1-biphenyl)(2-amino-1,1- |
| TFA | trifluoroacetic acid | | biphenyl-2-yl)palladium(II), CAS# |
| THF | tetrahydrofuran | | 1445085-55-1 |
| UPLC | ultra-performance liquid chromatography | | |

### Synthetic Preparation of the Compounds of the Invention

### Example 1. Preparation of Intermediates and Illustrative Compounds of the Invention

### 1.1. General Method 1: Azidation

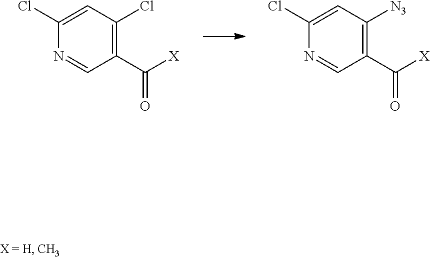

To a solution of 4,6-dichloropyridine-3-carbaldehyde or 1-(4,6-dichloro-3-pyridyl)ethanone (1.0 eq.) in dry DMF, cooled at 0° C. or at r.t., is added NaN₃ (1.0-1.1 eq.). The resulting mixture is stirred at r.t. for 1-2 h, then EtOAc is added, and the mixture is washed with sat. aq. NaHCO₃ solution and brine. The organic extract is dried over Na₂SO₄ and concentrated in vacuo to afford the expected intermediate as crude product.

### Illustrative Example of Method 1, Synthesis of Intermediate 1: 4-azido-6-chloronicotinaldehyde

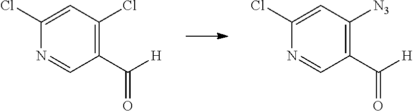 [SMILES: O=Cc1cnc(Cl)cc1Cl ; [N-]=[N+]=Nc1cc(Cl)ncc1C=O]

To an ice chilled solution of 4,6-dichloropyridine-3-carbaldehyde (528.0 mg, 3.0 mmol, 1.0 eq.) in dry DMF (5.0 mL), NaN₃ (204.8 mg, 6.30 mmol, 3.15 eq.) was added and the reaction mixture was stirred at r.t. After 1.5 h, EtOAc was added and the mixture was washed with sat. aq. NaHCO₃ solution and brine. The organic extract was dried over Na₂SO₄ and concentrated in vacuo to provide the desired intermediate.

### Another Illustrative Example of Method 1, Synthesis of Intermediate 2: 1-(4-azido-6-chloropyridin-3-yl)ethan-1-one

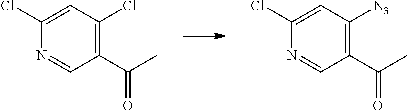

To a solution of 1-(4,6-dichloro-3-pyridyl)ethanone (5.0 g, 26.3 mmol, 1.0 eq.) in dry DMF (50 mL), NaN₃ (18.8 g, 28.9 mmol, 1.1 eq.) was added and the reaction mixture was stirred for 1.5 h at r.t. Upon complete conversion, reaction was stopped and quenched by addition of water and EtOAc. The aq. layer was extracted with EtOAc and the mixture was washed with sat. aq. solution of NaHCO₃ and brine. The organic extract was dried over MgSO₄ and concentrated in vacuo to provide the crude material which was suspended in MTBE, stirred for 1 h, filtered and dried to give the desired intermediate.

### 1.2. General methods 2: Cyclisation

### 1.2.1. Method 2-1: Cyclisation of Aldehyde

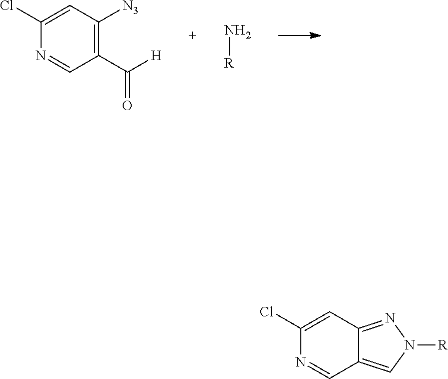

A solution of compound 4-azido-6-chloro-pyridine-3-carbaldehyde (1.0 eq.) and appropriate amine (1.0-1.1 eq.) in dry toluene is stirred at 80-100° C. for 1-5 h. Solvent is evaporated in vacuo and the obtained crude is purified by flash chromatography on silica gel.

Alternatively, the reaction is performed by combining 4-azido-6-chloro-pyridine-3-carbaldehyde (1.0 eq.) and appropriate amine (1.0 eq.) and heated without solvent in the sealed vial at 120° C. for 3-4 h.

### Illustrative Example of Method 2-1, Synthesis of Intermediate 3: 6-chloro-2-phenyl-2H-pyrazolo[4,3-c]pyridine

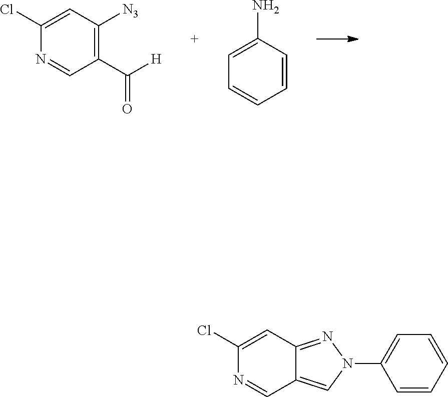 [SMILES: [N-]=[N+]=Nc1cc(Cl)ncc1C=O ; Nc1ccccc1 ; Clc1cc2nn(-c3ccccc3)cc2cn1]

A solution of 4-azido-6-chloro-pyridine-3-carbaldehyde (2.1 g, 11.5 mmol, 1.0 eq.) and aniline (1.05 mL, 11.5 mmol, 1.0 eq.) in dry toluene (20 mL) was stirred at 65° C. for 42 h. Solvent was evaporated in vacuo and the obtained crude was purified by flash chromatography on silica gel in the gradient of Hexane/EtOAc (0-50% EtOAc) to give the desired intermediate. 1.2.2. Method 2-2: Cyclisation of ketone

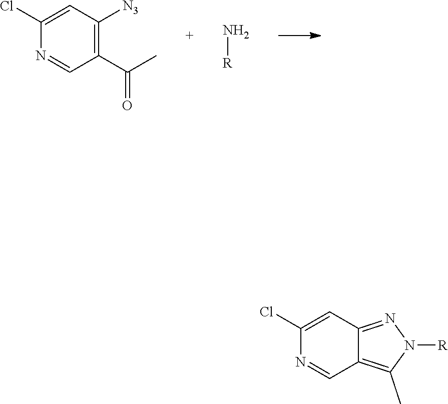

To a solution of 1-(4-azido-6-chloro-3-pyridyl)ethanone (1.0 eq.) in dry DCM at 0° C. is added amine (1.0 eq.), followed by titanium(IV) chloride (0.6 eq.) and TEA (3.0 eq.). The resulting mixture is stirred at 0° C. for 1 h, then at r.t. for 3-16 h. The reaction is stopped and quenched with cold NaHCO₃, sat. aq. solution. TiO₂ is filtered and solution is extracted with DCM. Organic layer is washed with brine and water, dried and evaporated. The obtained residue is purified by flash chromatography on silica gel.

### Illustrative Example of Method 2-2, Synthesis of Intermediate 17: 6-chloro-3-methyl-2-phenyl-2H-pyrazolo[4,3-c]pyridine

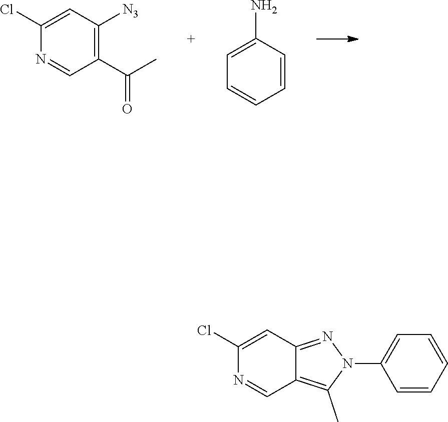 [SMILES: CC(=O)c1cnc(Cl)cc1N=[N+]=[N-] ; Nc1ccccc1 ; Cc1c2cnc(Cl)cc2nn1-c1ccccc1]

To a solution of 1-(4-azido-6-chloro-3-pyridyl)ethanone (51.7 mg, 0.263 mmol, 1.0 eq.) in dry DCM (1 mL) cooled to 0° C. were added aniline (0.0240 mL, 0.263 mmol, 1.0 eq.), titanium(IV) chloride (0.0173 mL, 0.158 mmol, 0.6 eq.) and TEA (0.110 mL, 0.789 mmol, 3.0 eq.). The resulting mixture was stirred at 0° C. for 1 h, then at r.t. for 3 h. The reaction was stopped and quenched with cold NaHCO₃, sat. aq. solution. TiO₂ was filtered and solution was extracted with DCM. Organic layer was washed with brine and water, dried and evaporated. The obtained residue was purified by flash chromatography on silica gel in the gradient of Hexane/EtOAc (0-50% EtOAc) to give desired intermediate.

### 1.3. General Method 3: Sulfonamide Synthesis

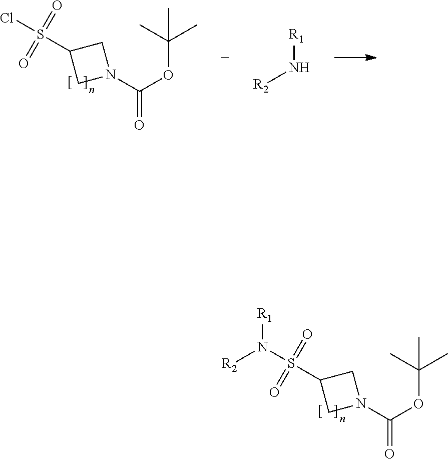

To a cooled (ice-bath) and stirred solution of appropriate amine (1.0-5.0 eq.) in suitable solvent (such as THF or DCM) with or without addition of the base such as TEA (3.0 eq.), tert-butyl 3-chlorosulfonylazetidine-1-carboxylate (1.0 eq.) is added. After addition the reaction mixture is allowed to warm to r.t. and stirred for 2-20 h. The expected intermediate may be isolated and, if desired, further purified by methods known to one skilled in the art.

### Illustrative Example of Method 3, Synthesis of Intermediate 22: tert-butyl 3-(N,N-dimethylsulfamoyl)azetidine-]-carboxylate

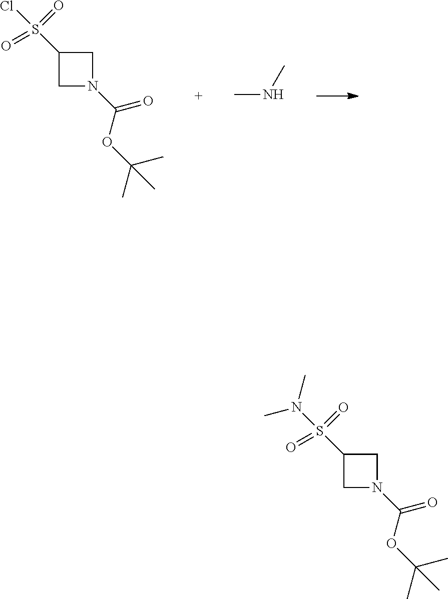

A solution of tert-butyl 3-chlorosulfonylazetidine-1-carboxylate (25.0 g, 97.8 mmol, 1.0 eq.) in dry THF (100 mL) was added dropwise to a cooled (ice-bath) and stirred solution of N-methylmethanamine (2.00 M, 245 mL, 489.0 mmol, 5.0 eq.) in THF. After addition the reaction mixture was allowed to warm to r.t. and stirred for 2 h. The reaction mixture was evaporated and the obtained residue dissolved in EtOAc (200 mL), washed with water (200 mL), then sat. aq. solution of NaHCO₃ (200 mL) and brine (200 mL). The organic layer was dried over anhydrous MgSO₄, filtered and evaporated to dryness to obtain the desired intermediate.

### Another Illustrative Example of Method 3, Synthesis of Intermediate 32: tert-butyl 3-(N,N-bis(4-methoxybenzyl) sulfamoyl)azetidine-1-carboxylate

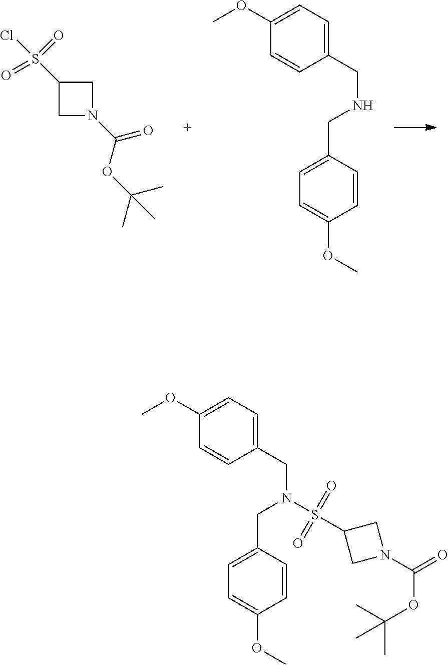

A solution of 1-(4-methoxyphenyl)-N-[(4-methoxyphenyl)methyl]methanamine (2.0 g, 7.77 mmol, 1.0 eq.) and TEA (2383 mg, 23.3 mmol, 3.0 eq.) in dry DCM (46.6 mL) was cooled to 0° C., then tert-butyl 3-chlorosulfonylazetidine-1-carboxylate (2208 mg, 8.55 mmol, 1.1 eq.) was added. The resulting mixture was left stirred at r.t. for 48 h. The reaction was diluted with DCM, washed with 5% Na₂CO₃ aq. solution, followed by 1 N HCl. The organic layer was dried and evaporated in vacuo affording crude product which was purified by flash chromatography on silica gel in the gradient of Hexane/EtOAc (0-20% EtOAc) to give desired intermediate.

### 1.4. General Method 4: Amine Deprotection

### 1.4.1. Method 4-1: Boc-Deprotection

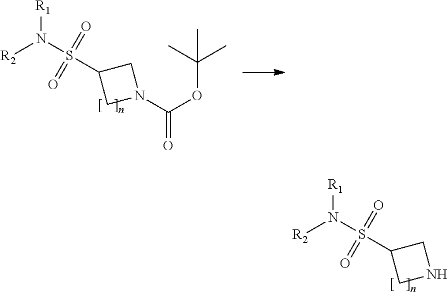

To a solution of the appropriate Boc-protected compound (1.0 eq.) in DCM is added TFA (5.0 eq.) and the reaction mixture is stirred at r.t. for 1-24 h to give the corresponding free amine product. The expected product may be isolated and, if desired further purified by methods known to one skilled in the art.

### Illustrative Example of Method 4-1, Synthesis Of Intermediate 21: N,N-dimethylazetidine-3-sulfonamide

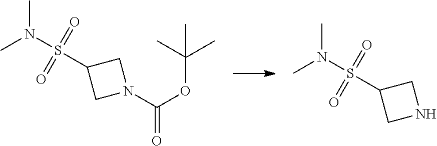

To a solution of tert-butyl 3-(dimethylsulfamoyl)azetidine-1-carboxylate (25.1 g, 94.0 mmol, 1.0 eq.) in dry DCM (350 mL) was added TFA (57.6 mL, 752.0 mmol, 8.0 eq.). The resulting mixture was left stirred at r.t. overnight. The reaction mixture was concentrated to dryness giving a TFA salt as a yellowish oil. The salt was divided in three equal portions. Each was then applied to a SCX column (preconditioned with MeOH) and eluted with MeOH followed by 7M ammonia in MeOH. Ammonia/MeOH fractions were combined and evaporated to dryness affording the desired intermediate.

### 1.4.2. Method 4-2: PMB-Deprotection

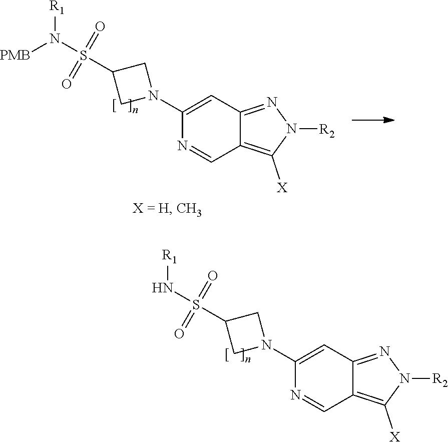

To a solution of the appropriate mono-PMB- or bis-PMB-protected compound (1.0 eq.) in DCM is added TFA (100-300 eq.) and the reaction mixture is stirred at r.t. 3-72 h to give the corresponding primary sulfonamide or monomethyl sulfonamide product. The expected product may be isolated and, if desired further purified by methods known to one skilled in the art.

### Illustrative Example of Method 4-2, Synthesis of Compound 56: N-methyl-]-(2-phenyl-2H-pyrazolo[4,3-c]pyridin-6-yl)azetidine-3-sulfonamide

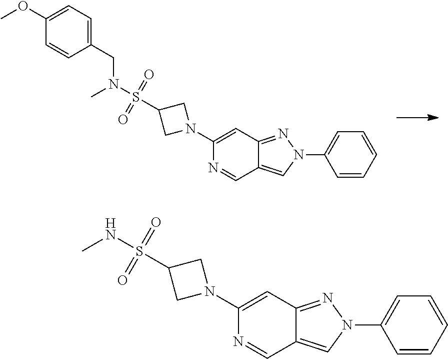 [SMILES: COc1ccc(CN(C)S(=O)(=O)C2CN(c3cc4nn(-c5ccccc5)cc4cn3)C2)cc1 ; CNS(=O)(=O)C1CN(c2cc3nn(-c4ccccc4)cc3cn2)C1]

To a solution of N-[(4-methoxyphenyl)methyl]-N-methyl-1-(2-phenylpyrazolo[4,3-c]pyridin-6-yl)azetidine-3-sulfonamide (46.9 mg, 0.101 mmol, 1.0 eq.) in DCM (2.0 mL) was added TFA (2.0 mL, 26.9 mmol, 266.0 eq.), and the mixture was stirred at r.t. for 3 h. The solvent was removed in vacuo to yield crude material which was submitted for preparative HPLC purification to obtain the desired compound.

### Another Illustrative Example of Method 4-2, Synthesis of Compound 41: 1-[2-(4-Fluorophenyl)pyrazolo[4,3-c]pyridin-6-yl]azetidine-3-sulfonamide

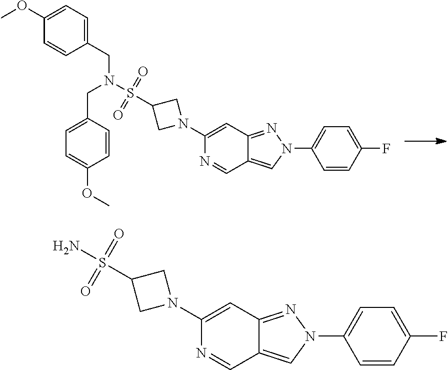

To a solution of 1-[2-(4-fluorophenyl)pyrazolo[4,3-c]pyridin-6-yl]-N,N-bis[(4-methoxyphenyl) methyl]azetidine-3-sulfonamide (42.0 mg, 0.0715 mmol, 1.0 eq.) in dry DCM (1.0 mL) was added TFA (1.00 mL, 13.1 mmol, 183.0 eq.). The resulting mixture was stirred at r.t. over weekend. The reaction mixture was diluted with DCM, then water was added and pH adjusted to 13-14 using 6N NaOH. Layers were separated. Desired product remained in water layer. The pH of the water layer was adjusted to pH ˜9 using 1N HCl. The resulting mixture was extracted with DCM and EtOAc. The precipitate was collected by filtration and combined with DCM and EtOAc extracts, solvent was evaporated to dryness affording crude product which was submitted to preparative HPLC purification and material was purified under acidic conditions. After purification desired compound was obtained.

### 1.5. General Method 5: Buchwald-Hartwig Reaction

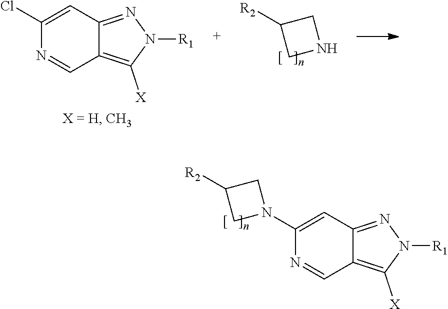

To a solution of the haloaryl derivative (1.0 eq.) in toluene, amine derivative (1.0-1.5 eq.), TEA (0.5 eq.) and KO^{t}Bu (3.0 eq.) are added. Reaction mixture is degassed with Argon, XPhos Pd G3 (0.1 eq.) is added and the solution is again degassed by bubbling Argon. The reaction mixture is heated to 80° C. and stirred for 0.5-20 h. The expected product may be isolated and, if desired further purified by methods known to one skilled in the art.

### Illustrative Example of Method 5, Synthesis of Compound 1: N,N-dimethyl-]-(2-phenylpyrazolo[4,3-c]pyridin-6-yl)azetidine-3-sulfonamide

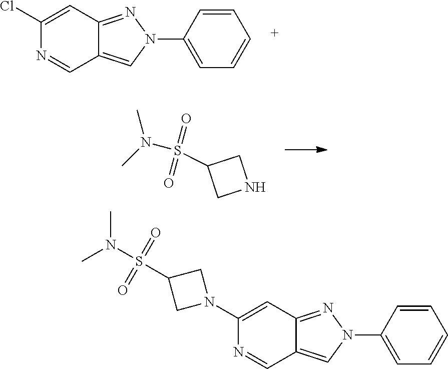

To a solution of 6-chloro-2-phenyl-pyrazolo[4,3-c]pyridine (1.84 g, 8.01 mmol, 1 eq.) in toluene (80.0 mL), N,N-dimethylazetidine-3-sulfonamide (1.32 g, 8.01 mmol, 1 eq.), TEA (0.55 ml, 4.01 mmol, 0.5 eq.), and KO^{t}Bu (2.70 g, 24.0 mmol, 3 eq.) were added. Reaction mixture was degassed with Argon, XPhos Pd G3 (0.678 g, 0.80 mmol, 0.1 eq.) ws added and the solution was again degassed by bubbling Argon. The reaction mixture was heated to 80° C. and stirred overnight. The reaction mixture was filtrated over pad of Celite and solvent was removed in vacuo. The residue was purified by flash chromatography on silica gel in the gradient of DCM/MeOH (0-5% MeOH) to give desired compound.

### 1.6. General Method 6: SNAr

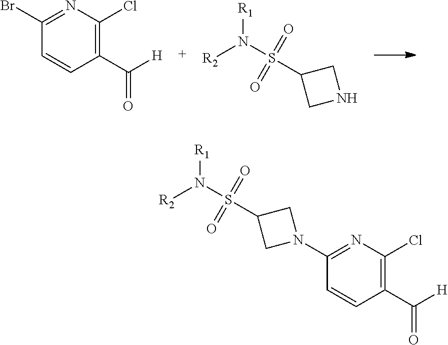

To a solution of 6-bromo-2-chloro-pyridine-3-carbaldehyde (1.0 eq) in dry DMF potassium carbonate (1.4-2.0 eq) is added, followed by appropriate azetidine-3-sulfonamide (1.0-1.1 eq). The resulting mixture is heated and stirred at 60° C. for 2-5 h. Upon complete conversion the reaction mixture is cooled down to r.t., then water and EtOAc are added and the mixture is washed with sat. aq.

NaHCO₃ and water. The organic layer is dried over Na₂SO₄ and concentrated in vacuo to provide crude product which is purified by flash chromatography on silica gel.

### Illustrative Example of Method 6, Synthesis of Intermediate 47: 1-(6-chloro-5-formyl-2-pyridyl)-N,N-dimethyl-azetidine-3-sulfonamide

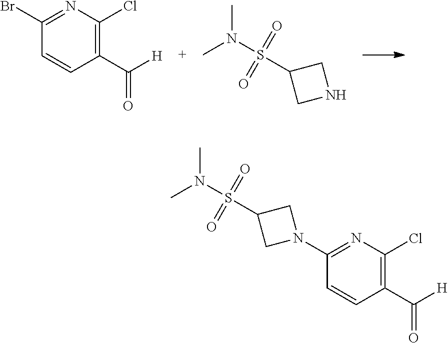

To a solution of 6-bromo-2-chloro-pyridine-3-carbaldehyde (220 mg, 1.00 mmol, 1.0 eq.) in dry DMF (5.00 mL) potassium carbonate (193 mg, 1.40 mmol, 1.4 eq.) was added, followed by N,N-dimethylazetidine-3-sulfonamide (164 mg, 1.00 mmol, 1.0 eq.). The resulting mixture was heated and stirred at 60° C. for 2 h. Upon complete conversion, the reaction mixture was cooled down to r.t. and quenched with water, then EtOAc was added and the mixture was washed with sat. aq. solution of NaHCO₃ and water. The organic extract was dried over Na₂SO₄ and concentrated in vacuo to provide crude product which was purified by flash chromatography on silica gel in the gradient of Hexane/EtOAc (0-50% EtOAc) to give the desired intermediate.

### 1.7. General Method 7: Chlorination

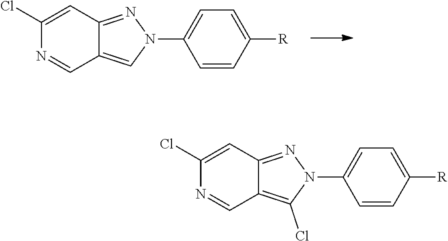

To a solution of appropriate pyrazolo[4,3-c]pyridine (1.0 eq.) in ACN is added N-chlorosuccinimide (1.0 eq.). Reaction mixture is stirred overnight at 50° C. The reaction is stopped and solvent evaporated. The obtained residue is purified by flash chromatography on silica gel.

### Illustrative Example of Method 7, Synthesis of Intermediate 42: 3,6-dichloro-2-(4-fluorophenyl)pyrazolo[4,3-c]pyridine

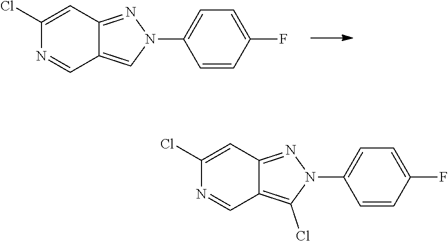

To a solution of 6-chloro-2-(4-fluorophenyl)pyrazolo[4,3-c]pyridine (500 mg, 2.02 mmol, 1.0 eq.) in ACN (18.00 mL) was added N-chlorosuccinimide (270 mg, 2.02 mmol, 1.0 eq.). Reaction mixture was stirred overnight at 50° C. The reaction was stopped and solvent evaporated. The obtained residue was purified by flash chromatography on silica gel in the gradient of Hexane/EtOAc (0-50% EtOAc) to give the desired intermediate.

### 1.8. General Method 8: Suzuki-Miyaura Reaction

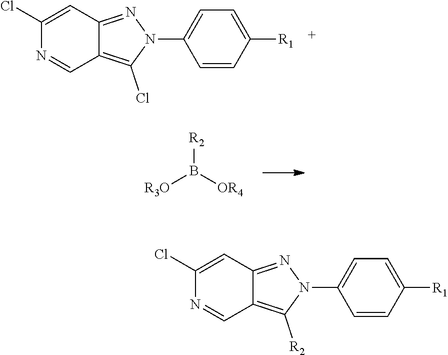

To a solution of appropriate dichloropyrazolo[4,3-c]pyridine (1.0 eq.) in 1,4-dioxane is added appropriate boronic acid (or boronic ester) (2.0-3.0 eq.) followed by sat. aq. solution Na₂CO₃ and tetrakis(triphenylphosphine)palladium(0) (0.05 eq.). Reaction mixture is degassed with Argon and stirred under microwave irradiation at 120° C. for 15 min or overnight at 75° C. Upon complete conversion, reaction is cooled down to r.t., then EtOAc is added and reaction washed with brine and water. Organics are dried over Na₂SO₄ and concentrated in vacuo to provide crude product which is purified by flash chromatography on silica gel.

### Illustrative Example of Method 8, Synthesis of Intermediate 46: 6-chloro-2-(4-fluorophenyl)-3-(prop-1-en-2-yl)-2H-pyrazolo[4,3-c]pyridine

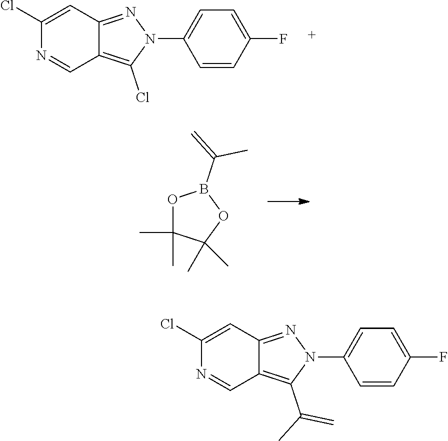

To a solution of 3,6-dichloro-2-(4-fluorophenyl)pyrazolo[4,3-c]pyridine (56.4 mg, 0.200 mmol, 1.0 eq.) in 1,4-dioxane (2.00 mL) was added 2-isopropenyl-4,4,5,5-tetramethyl-1,3,2-dioxaborolane (0.0764 mL, 0.400 mmol, 2.0 eq.) followed by sat. aq. solution of Na₂CO₃ (0.250 mL) and tetrakis(triphenylphosphine)palladium(0) (11.6 mg, 0.0100 mmol, 0.05 eq.). Reaction mixture was degassed with Argon and stirred under microwave irradiation at 120° C. for 15 min. Upon complete conversion, reaction was stopped, EtOAc was added and washed with brine and water. Organics were dried over Na₂SO₄ and concentrated in vacuo to provide crude material which was purified by flash chromatography on silica gel in the gradient of Hexane/EtOAc (0-50% EtOAc) to give the desired intermediate.

### 1.9. General Method 9: Hydrogenation

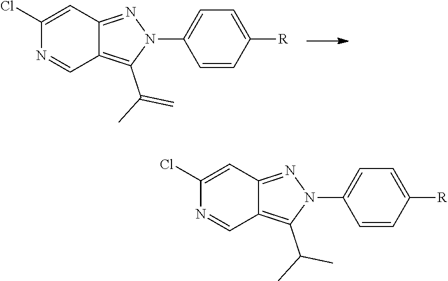

To a solution of appropriate dichloropyrazolo[4,3-c]pyridine (1.0 eq.) in dry in MeOH is added Palladium, 10% on carbon (0.01-0.05 eq.). The resulting mixture is stirred under the atmosphere of hydrogen for 2 h at r.t. Upon complete conversion, reaction mixture is filtered through celite and solvent evaporated to afford crude material which is purified by flash chromatography on silica gel.

### Illustrative Example of Method 9, Synthesis of Intermediate 45: 6-chloro-2-(4-fluorophenyl)-3-isopropyl-2H-pyrazolo[4,3-c]pyridine

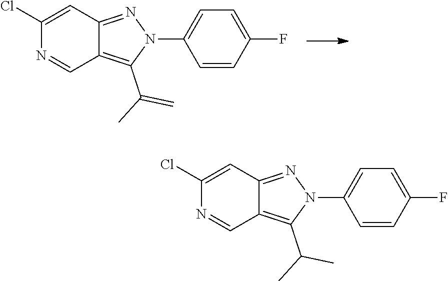 [SMILES: C=C(C)c1c2cnc(Cl)cc2nn1-c1ccc(F)cc1 ; CC(C)c1c2cnc(Cl)cc2nn1-c1ccc(F)cc1]

To a solution of 6-chloro-2-(4-fluorophenyl)-3-isopropenyl-pyrazolo[4,3-c]pyridine (48.0 mg, 0.167 mmol, 1.0 eq.) in MeOH dry (0.5 mL) was added Palladium, 10% on carbon (10.0, 1.78 mg, 0.00167 mmol, 0.01 eq.). The resulting mixture was stirred under the atmosphere of hydrogen for 1 h at r.t. after which additional amount of Palladium, 10% on carbon (10.0 00 8.88 mg, 0.00834 mmol, 0.05 eq.) was added and mixture was stirred under the atmosphere of hydrogen for further 1 h at r.t. Upon complete conversion, reaction mixture was filtered through Celite and solvent evaporated to afford crude material which was purified by flash chromatography on silica gel in the gradient of Hexane/EtOAc (0-50% EtOAc) to give the desired intermediate.

TABLE II
Intermediates used towards the compounds of the invention.

| | | | | | | MS |
|---|---|---|---|---|---|---|
| Int # | Structure | Name | SM | Mtd | MW | Mes′d |
| 1 | 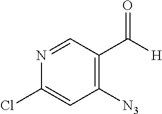 [SMILES: [N-]=[N+]=Nc1cc(Cl)ncc1C=O] | 4-azido-6-chloro nicotinaldehyde | CAS# 1060811- 62-2 | 1 | 182.6 | 155.0 |
| 2 | 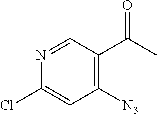 | 1-(4-azido-6- chloropyridin-3- yl)ethan-1-one | CAS# 887573- 44-6 | 1 | 196.6 | 169.0 |
| 3 | 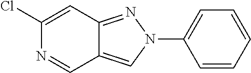 [SMILES: Clc1cc2nn(-c3ccccc3)cc2cn1] | 6-chloro-2-phen- yl-2H-pyrazolo [4,3-c]pyridine | Int 1 & CAS# 62-53-3 | 2-1 | 229.7 | 230.0 |
| 4 | 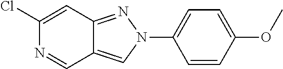 [SMILES: COc1ccc(-n2cc3cnc(Cl)cc3n2)cc1] | 6-chloro-2-(4- methoxyphenyl)- 2H-pyrazolo[4,3- c]pyridine | Int 1 & CAS# 104-94-9 | 2-1 | 259.7 | 260.2 |
| 5 | 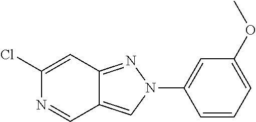 [SMILES: COc1cccc(-n2cc3cnc(Cl)cc3n2)c1] | 6-chloro-2-(3- methoxyphenyl)- 2H-pyrazolo[4,3- c]pyridine | Int 1 & CAS# 536-90-3 | 2-1 | 259.7 | 260.6 |
| 6 | 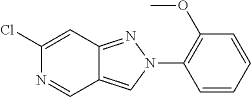 [SMILES: COc1ccccc1-n1cc2cnc(Cl)cc2n1] | 6-chloro-2-(2- methoxyphenyl)- 2H-pyrazolo[4,3- c]pyridine | Int 1 & CAS# 90-04-0 | 2-1 | 259.7 | 260.5 |
| 7 | 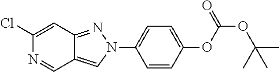 [SMILES: CC(C)(C)OC(=O)Oc1ccc(-n2cc3cnc(Cl)cc3n2)cc1] | tert-butyl (4-(6- chloro-2H-pyr- azolo[4,3-c]pyr- idin-2-yl)phenyl)- carbonate | Int 1 & CAS# 95932-39-1 | 2-1 | 345.8 | 346.0 |
| 8 | 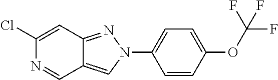 [SMILES: FC(F)(F)Oc1ccc(-n2cc3cnc(Cl)cc3n2)cc1] | 6-chloro-2-(4- (trifluorometh- oxy)phenyl)-2H- pyrazolo[4,3-c] pyridine | Int 1 & CAS# 461-82-5 | 2-1 | 313.7 | 314.0 |
| 9 | 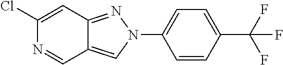 [SMILES: FC(F)(F)c1ccc(-n2cc3cnc(Cl)cc3n2)cc1] | 6-chloro-2-(4- (trifluorometh- yl)phenyl)-2H- pyrazolo[4,3-c] | Int 1 & CAS# 455-14-1 | 2-1 | 297.7 | 298.0 |
| 10 | 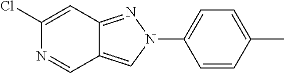 [SMILES: Cc1ccc(-n2cc3cnc(Cl)cc3n2)cc1] | 6-chloro-2-(p-tol- yl)-2H-pyrazolo [4,3-c]pyridine | Int 1 & CAS# 106-49-0 | 2-1 | 243.7 | 244.0 |
| 11 | 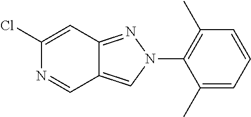 [SMILES: Cc1cccc(C)c1-n1cc2cnc(Cl)cc2n1] | 6-chloro-2-(2,6- dimethylphenyl)- 2H-pyrazolo[4,3- c]pyridine | Int 1 & CAS# 87-62-7 | 2-1 | 257.7 | 258.5 |
| 12 | 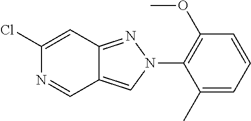 [SMILES: COc1cccc(C)c1-n1cc2cnc(Cl)cc2n1] | 6-chloro-2-(2- methoxy-6-meth- ylphenyl)-2H- pyrazolo[4,3-c] pyridine | Int 1 & CAS# 50868-73-0 | 2-1 | 273.7 | 274.5 |
| 13 | 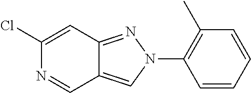 [SMILES: Cc1ccccc1-n1cc2cnc(Cl)cc2n1] | 6-chloro-2-(o-tol- yl)-2H-pyrazolo [4,3-c]pyridine | Int 1 & CAS# 95-53-4 | 2-1 | 243.7 | 244.5 |
| 14 | 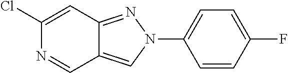 [SMILES: Fc1ccc(-n2cc3cnc(Cl)cc3n2)cc1] | 6-chloro-2-(4- fluorophenyl)- 2H-pyrazolo[4,3- c]pyridine | Int 1 & CAS# 371-40-4 | 2-1 | 247.7 | 248.1 |
| 15 | 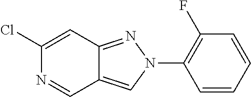 [SMILES: Fc1ccccc1-n1cc2cnc(Cl)cc2n1] | 6-chloro-2-(2- fluorophenyl)-2H- pyrazolo[4,3-c] pyridine | Int 1 & CAS# 348-54-9 | 2-1 | 247.7 | 248.0 |
| 16 | 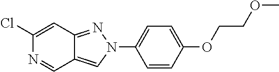 [SMILES: COCCOc1ccc(-n2cc3cnc(Cl)cc3n2)cc1] | 6-chloro-2-(4- (2-methoxyeth- oxy)phenyl)-2H- pyrazolo[4,3-c] pyridine | Int 1 & CAS# 33311-29-4 | 2-1 | 303.7 | 304.1 |
| 17 | 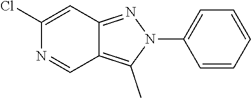 [SMILES: Cc1c2cnc(Cl)cc2nn1-c1ccccc1] | 6-chloro-3-meth- yl-2-phenyl-2H- pyrazolo[4,3-c] pyridine | Int 2 & CAS# 62-53-3 | 2-2 | 243.7 | 244.0 |
| 18 | 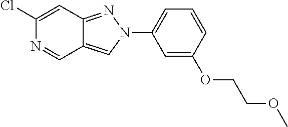 [SMILES: COCCOc1cccc(-n2cc3cnc(Cl)cc3n2)c1] | 6-chloro-2-(3-(2- methoxyethoxy)- phenyl)-2H-pyr- azolo[4,3-c] pyridine | Int 2 & CAS# 110178- 35-3 | 2-1 | 303.7 | 304.0 |
| 19 | 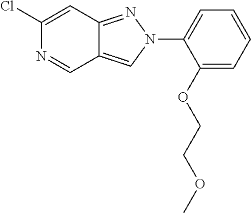 [SMILES: COCCOc1ccccc1-n1cc2cnc(Cl)cc2n1] | 6-chloro-2-(2-(2- methoxyethoxy)- phenyl)-2H- pyrazolo[4,3-c] pyridine | Int 2 & CAS# 110178- 35-3 | 2-1 | 303.7 | 304.0 |
| 20 | 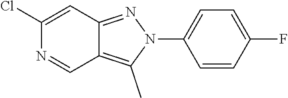 [SMILES: Cc1c2cnc(Cl)cc2nn1-c1ccc(F)cc1] | 6-chloro-2-(4- fluorophenyl)- 3-methyl-2H- pyrazolo[4,3-c] pyridine | Int 2 & CAS# 371-40-4 | 2-2 | 261.7 | 262.1 |
| 21 | 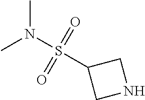 | N,N-dimethyl- azetidine-3- sulfonamide | Int 22 | 4-1 | 164.2 | 165.1 |
| 22 | 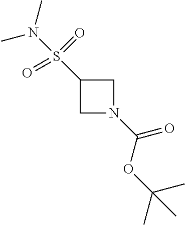 [SMILES: CN(C)S(=O)(=O)C1CN(C(=O)OC(C)(C)C)C1] | tert-butyl 3-(N,N- dimethylsulfam- oyl)azetidine-1- carboxylate | CAS# 1310732- 18-3 & CAS# 124-40-3 | 3 | 264.3 | 209.1 |
| 23 | 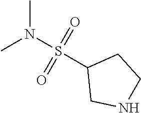 | N,N-dimethylpyr- rolidine-3-sulfon- amide | Int 24 | 4-1 | 178.3 | 179.1 |
| 24 | 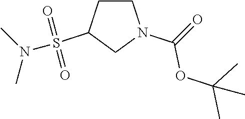 [SMILES: CN(C)S(=O)(=O)C1CCN(C(=O)OC(C)(C)C)C1] | tert-butyl 3-(N,N- dimethylsulfam- oyl)pyrrolidine- 1-carboxylate | CAS# 935845- 20-8 & CAS# 124-40-3 | 3 | 278.4 | 223.0 |
| 25 | 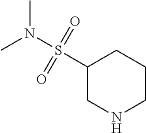 | N,N-dimethyl- piperidine-3- sulfonamide | Int 26 | 4-1 | 192.3 | 193.1 |
| 26 | 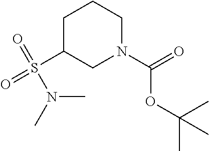 [SMILES: CN(C)S(=O)(=O)C1CCCN(C(=O)OC(C)(C)C)C1] | tert-butyl 3-(N,N- dimethylsulfam- oyl)piperidine- 1-carboxylate | CAS# 124- 40-3 & CAS# 1260664- 44-5 | 3 | 292.4 | 237.1 |
| 27 | 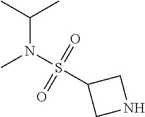 | N-isopropyl-N- methylazetidine- 3-sulfonamide | Int 28 | 4-1 | 192.3 | 193.1 |
| 28 | 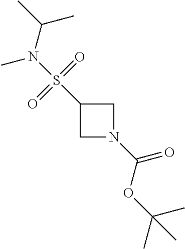 [SMILES: CC(C)N(C)S(=O)(=O)C1CN(C(=O)OC(C)(C)C)C1] | tert-butyl 3-(N- isopropyl-N- methylsulfam- oyl)azetidine-1- carboxylate | CAS# 1310732- 18-3 & CAS# 4747-21-1 | 3 | 292.4 | 237.2 |
| 29 | 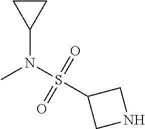 | N-cyclopropyl-N- methylazetidine- 3-sulfonamide | Int 30 | 4-1 | 190.3 | 191.1 |
| 30 | 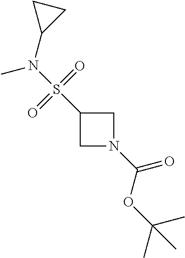 [SMILES: CN(C1CC1)S(=O)(=O)C1CN(C(=O)OC(C)(C)C)C1] | tert-butyl 3-(N- cyclopropyl-N- methylsulfam- oyl)azetidine-1- carboxylate | CAS# 1310732- 18-3 & CAS# 5163-20-2 | 3 | 290.4 | 235.1 |
| 31 | 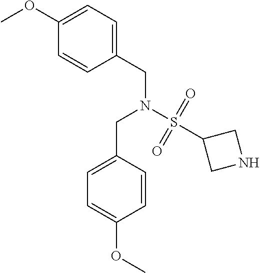 [SMILES: COc1ccc(CN(Cc2ccc(OC)cc2)S(=O)(=O)C2CNC2)cc1] | N,N-bis(4-meth- oxybenzyl)azeti- dine-3-sulfon- amide | Int 32 | 4-1 | 376.5 | 377.2 |
| 32 | 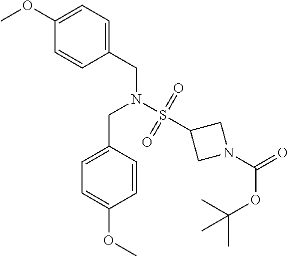 [SMILES: COc1ccc(CN(Cc2ccc(OC)cc2)S(=O)(=O)C2CN(C(=O)OC(C)(C)C)C2)cc1] | tert-butyl 3-(N,N- bis(4-methoxy- benzyl)sulfam- oyl)azetidine-1- carboxylate | CAS# 1310732- 18-3 & CAS# 17061-62-0 | 3 | 476.6 | 377.1 |
| 33 | 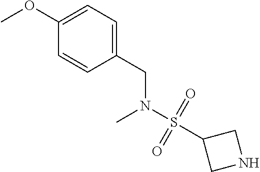 [SMILES: COc1ccc(CN(C)S(=O)(=O)C2CNC2)cc1] | N-(4-methoxy- benzyl)-N-meth- ylazetidine-3- sulfonamide | Int 33 | 4-1 | 270.4 | 271.1 |
| 34 | 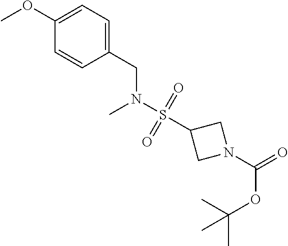 [SMILES: COc1ccc(CN(C)S(=O)(=O)C2CN(C(=O)OC(C)(C)C)C2)cc1] | tert-butyl 3-(N-(4- methoxybenzyl)- N-methylsulfam- oyl)azetidine-1- carboxylate | CAS# 1310732- 18-3 & CAS# 702-24-9 | 3 | 370.5 | 271.1 |
| 35 | 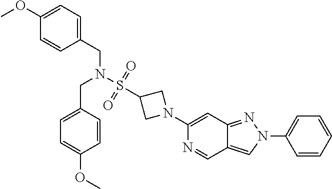 [SMILES: COc1ccc(CN(Cc2ccc(OC)cc2)S(=O)(=O)C2CN(c3cc4nn(-c5ccccc5)cc4cn3)C2)cc1] | N,N-bis(4-meth- oxybenzyl)-1-(2- phenyl-2H-pyr- azolo[4,3-c]pyr- idin-6-yl)azeti- dine-3-sulfon- amide | Int 31 & Int 3 | 5 | 569.7 | 570.4 |
| 36 | 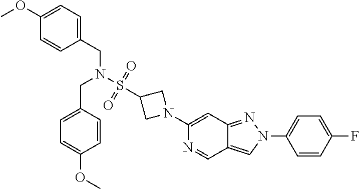 [SMILES: COc1ccc(CN(Cc2ccc(OC)cc2)S(=O)(=O)C2CN(c3cc4nn(-c5ccc(F)cc5)cc4cn3)C2)cc1] | 1-(2-(4-fluoro- phenyl)-2H-pyr- azolo[4,3-c]pyr- idin-6-yl)-N,N- bis(4-methoxy- benzyl)azetidine- 3-sulfonamide | Int 31 & Int 14 | 5 | 587.7 | 588.1 |
| 37 | 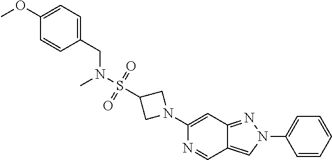 [SMILES: COc1ccc(CN(C)S(=O)(=O)C2CN(c3cc4nn(-c5ccccc5)cc4cn3)C2)cc1] | N-(4-methoxy- benzyl)-N-meth- yl-1-(2-phenyl- 2H-pyrazolo[4,3- c]pyridin-6-yl)- azetidine-3- sulfonamide | Int 33 & Int 3 | 5 | 463.6 | 464.2 |
| 38 | 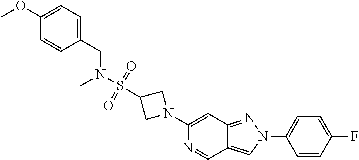 [SMILES: COc1ccc(CN(C)S(=O)(=O)C2CN(c3cc4nn(-c5ccc(F)cc5)cc4cn3)C2)cc1] | 1-(2-(4-fluoro- phenyl)-2H- pyrazolo[4,3-c] pyridin-6-yl)-N- (4-methoxybenz- yl)-N-methyl- azetidine-3- sulfonamide | Int 33 & Int 14 | 5 | 481.5 | 482.2 |
| 39 | 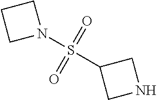 [SMILES: O=S(=O)(C1CNC1)N1CCC1] | 1-(azetidin-3-yl- sulfonyl)azetidine | Int 40 | 4-1 | 176.2 | 177.2 |
| 40 | 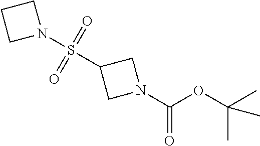 [SMILES: CC(C)(C)OC(=O)N1CC(S(=O)(=O)N2CCC2)C1] | tert-butyl 3- (azetidin-1-ylsul- fonyl)azetidine- 1-carboxylate | CAS# 1310732- 18-3 & CAS# 503-29-7 | 3 | 276.4 | 221.0 |
| 41 | 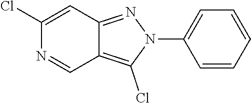 [SMILES: Clc1cc2nn(-c3ccccc3)c(Cl)c2cn1] | 3,6-dichloro-2- phenyl-2H- pyrazolo[4,3-c] pyridine | Int 3 | 7 | 264.1 | 266.0 |
| 42 | 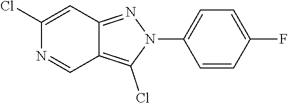 [SMILES: Fc1ccc(-n2nc3cc(Cl)ncc3c2Cl)cc1] | 3,6-dichloro-2- (4-fluorophenyl)- 2H-pyrazolo[4,3- c]pyridine | Int 14 | 7 | 282.1 | 283.9 |
| 43 | 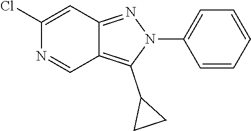 [SMILES: Clc1cc2nn(-c3ccccc3)c(C3CC3)c2cn1] | 6-chloro-3-cyclo- propyl-2-phenyl- 2H-pyrazolo[4,3- c]pyridine | Int 41 & CAS# 411235- 57-9 | 8 | 269.7 | 270.1 |
| 44 | 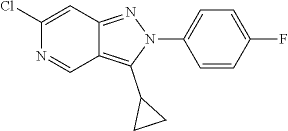 [SMILES: Fc1ccc(-n2nc3cc(Cl)ncc3c2C2CC2)cc1] | 6-chloro-3-cyclo- propyl-2-(4- fluorophenyl)- 2H-pyrazolo[4,3- c]pyridine | Int 42 & CAS# 411235- 57-9 | 8 | 287.7 | 288.0 |
| 45 | 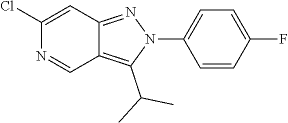 [SMILES: CC(C)c1c2cnc(Cl)cc2nn1-c1ccc(F)cc1] | 6-chloro-2-(4- fluorophenyl)-3- isopropyl-2H- pyrazolo[4,3-c] pyridine | Int 46 | 9 | 289.7 | 290.1 |
| 46 | 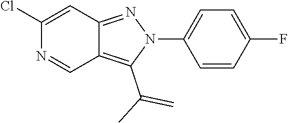 [SMILES: C=C(C)c1c2cnc(Cl)cc2nn1-c1ccc(F)cc1] | 6-chloro-2-(4- fluorophenyl)-3- (prop-1-en-2-yl)- 2H-pyrazolo[4,3- c]pyridine | Int 42 & CAS# 126726- 62-3 | 8 | 287.7 | 288.0 |
| 47 | 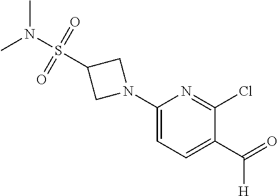 [SMILES: CN(C)S(=O)(=O)C1CN(c2ccc(C=O)c(Cl)n2)C1] | 1-(6-chloro-5- formylpyridin- 2-yl)-N,N-dimeth- ylazetidine-3- sulfonamide | CAS# 1125410- 08-3 & Int 21 | 6 | 303.8 | 304.0 |
| 49 | 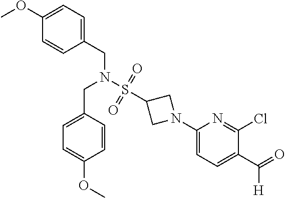 [SMILES: COc1ccc(CN(Cc2ccc(OC)cc2)S(=O)(=O)C2CN(c3ccc(C=O)c(Cl)n3)C2)cc1] | 1-(6-chloro-5- formylpyridin-2- yl)-N,N-bis(4- methoxybenzyl)- azetidine-3- sulfonamide | CAS# 1125410- 08-3 & Int 32 | 6 | 516.00 | 516.2 |
| 50 | 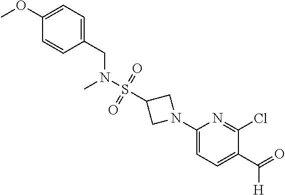 [SMILES: COc1ccc(CN(C)S(=O)(=O)C2CN(c3ccc(C=O)c(Cl)n3)C2)cc1] | 1-(6-chloro-5- formylpyridin-2- yl)-N-(4-meth- oxybenzyl)-N- methylazetidine- 3-sulfonamide | CAS# 1125410- 08-3 & Int 33 | 6 | 409.9 | 410.2 |
| 51 | 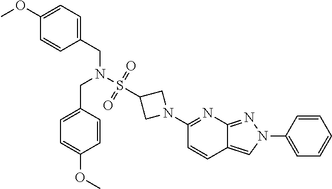 [SMILES: COc1ccc(CN(Cc2ccc(OC)cc2)S(=O)(=O)C2CN(c3ccc4cn(-c5ccccc5)nc4n3)C2)cc1] | N,N-bis(4-meth- oxybenzyl)-1-(2- phenyl-2H-pyr- azolo[3,4-b] pyridin-6-yl)- azetidine-3- sulfonamide | Int 49 & CAS# 62-53-3 | 1 & 2-1 | 569.7 | 570.3 |
| 52 | 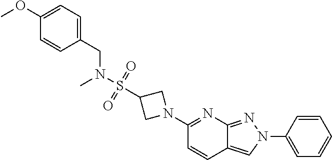 [SMILES: COc1ccc(CN(C)S(=O)(=O)C2CN(c3ccc4cn(-c5ccccc5)nc4n3)C2)cc1] | N-(4-methoxy- benzyl)-N-methyl- 1-(2-phenyl-2H- pyrazolo[3,4-b] pyridin-6-yl)- azetidine-3- sulfonamide | Int 50 & CAS# 62-53-3 | 1 & 2-1 | 463.6 | 464.2 |
| 53 | 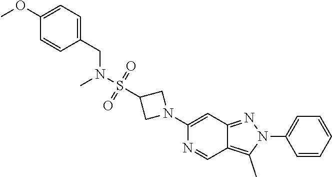 [SMILES: COc1ccc(CN(C)S(=O)(=O)C2CN(c3cc4nn(-c5ccccc5)c(C)c4cn3)C2)cc1] | N-(4-methoxy- benzyl)-N-meth- yl-1-(3-methyl- 2-phenyl-2H- pyrazolo[4,3- c]pyridin-6- yl)azetidine-3- sulfonamide | Int 33 & Int 17 | 5 | 477.6 | 478.1 |
| 54 | 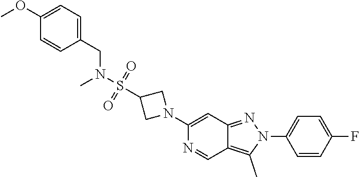 [SMILES: COc1ccc(CN(C)S(=O)(=O)C2CN(c3cc4nn(-c5ccc(F)cc5)c(C)c4cn3)C2)cc1] | 1-(2-(4-fluoro- phenyl)-3-meth- yl-2H-pyrazolo [4,3-c]pyridin-6- yl)-N-(4-meth- oxybenzyl)-N- methylazetidine- 3-sulfonamide | Int 33 & Int 20 | 5 | 495.6 | 496.1 |
| 55 | 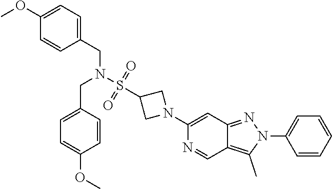 [SMILES: COc1ccc(CN(Cc2ccc(OC)cc2)S(=O)(=O)C2CN(c3cc4nn(-c5ccccc5)c(C)c4cn3)C2)cc1] | N,N-bis(4-meth- oxybenzyl)-1-(3- methyl-2-phenyl- 2H-pyrazolo[4,3- c]pyridin-6-yl)- azetidine-3- sulfonamide | Int 31 & Int 17 | 5 | 583.7 | 584.2 |
| 56 | 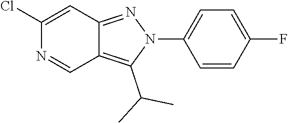 [SMILES: CC(C)c1c2cnc(Cl)cc2nn1-c1ccc(F)cc1] | 1-(2-(4-fluoro- phenyl)-3-methyl- 2H-pyrazolo[4,3- c]pyridin-6-yl)- N,N-bis(4-meth- oxybenzyl)azeti- dine-3-sulfon- amide | Int 31 & Int 20 | 5 | 601.7 | 602.1 |

SM = Starting Material, Mtd = Method, MS Mes′d = Measured mass

TABLE III
Illustrative compounds of the invention

| | | | Starting | | MW | MW |
|---|---|---|---|---|---|---|
| Cpd | Structure | Name | material | Mtd | (calc) | (obs) |
| 1 | 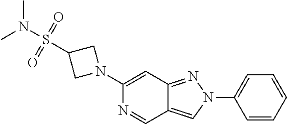 [SMILES: CN(C)S(=O)(=O)C1CN(c2cc3nn(-c4ccccc4)cc3cn2)C1] | N,N-dimethyl-1-(2- phenyl-2H-pyrazolo [4,3-c]pyridin-6-yl)- azetidine-3-sulfon- amide | Int 3 & Int 21 | 5 | 357.4 | 358.0 |
| 2 | 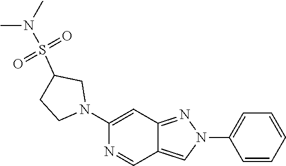 [SMILES: CN(C)S(=O)(=O)C1CCN(c2cc3nn(-c4ccccc4)cc3cn2)C1] | N,N-dimethyl-1-(2- phenyl-2H-pyrazolo [4,3-c]pyridin-6-yl)- pyrrolidine-3-sulfon- amide | Int 3 & Int 23 | 5 | 371.5 | 372.2 |
| 3 | 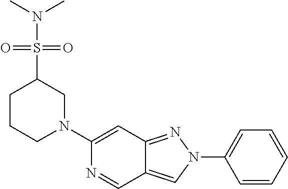 [SMILES: CN(C)S(=O)(=O)C1CCCN(c2cc3nn(-c4ccccc4)cc3cn2)C1] | N,N-dimethyl-1-(2- phenyl-2H-pyrazolo [4,3-c]pyridin-6-yl)- piperidine-3-sulfon- amide | Int 3 & Int 25 | 5 | 385.5 | 386.3 |
| 5 | 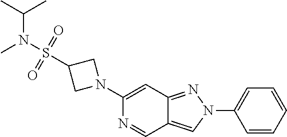 [SMILES: CC(C)N(C)S(=O)(=O)C1CN(c2cc3nn(-c4ccccc4)cc3cn2)C1] | N-isopropyl-N- methyl-1-(2-phenyl- 2H-pyrazolo[4,3-c] pyridin-6-yl)azeti- dine-3-sulfonamide | Int 3 & Int 27 | 5 | 385.5 | 386.3 |
| 7 | 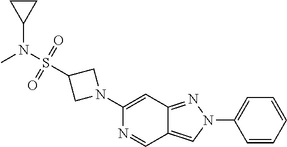 [SMILES: CN(C1CC1)S(=O)(=O)C1CN(c2cc3nn(-c4ccccc4)cc3cn2)C1] | N-cyclopropyl-N- methyl-1-(2-phenyl- 2H-pyrazolo[4,3-c] pyridin-6-yl)azeti- dine-3-sulfonamide | Int 3 & Int 29 | 5 | 383.5 | 384.3 |
| 8 | 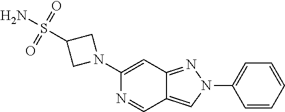 [SMILES: NS(=O)(=O)C1CN(c2cc3nn(-c4ccccc4)cc3cn2)C1] | 1-(2-phenyl-2H-pyr- azolo[4,3-c]pyridin- 6-yl)azetidine-3- sulfonamide | Int 3 & Int 31 | 4-2 | 329.4 | 330.2 |
| 9 | 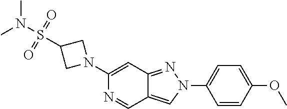 | 1-(2-(4-methoxy- phenyl)-2H-pyr- azolo[4,3-c]pyridin- 6-yl)-N,N-dimethyl- azetidine-3-sulfon- amide | Int 3 & Int 21 | 5 | 387.5 | 388.3 |
| 10 | 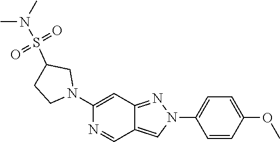 | 1-(2-(4-methoxy- phenyl)-2H-pyr- azolo[4,3-c]pyridin- 6-yl)-N,N-dimethyl- pyrrolidine-3-sulfon- amide | Int 4 & Int 23 | 5 | 401.5 | 402.3 |
| 14 | 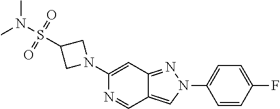 [SMILES: CN(C)S(=O)(=O)C1CN(c2cc3nn(-c4ccc(F)cc4)cc3cn2)C1] | 1-(2-(4-fluorophen- yl)-2H-pyrazolo[4,3- c]pyridin-6-yl)-N,N- dimethylazetidine-3- sulfonamide | Int 3 & Int 14 | 5 | 375.4 | 376.3 |
| 15 | 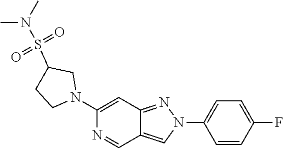 [SMILES: CN(C)S(=O)(=O)C1CCN(c2cc3nn(-c4ccc(F)cc4)cc3cn2)C1] | 1-(2-(4-fluorophen- yl)-2H-pyrazolo[4,3- c]pyridin-6-yl)-N,N- dimethylpyrrolidine- 3-sulfonamide | Int 23 & Int 14 | 5 | 389.5 | 390.3 |
| 16 | 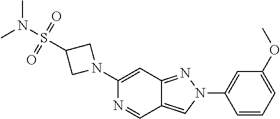 [SMILES: COc1cccc(-n2cc3cnc(N4CC(S(=O)(=O)N(C)C)C4)cc3n2)c1] | 1-(2-(3-methoxy- phenyl)-2H-pyrazolo [4,3-c]pyridin-6-yl)- N,N-dimethylazeti- dine-3-sulfonamide | Int 21 & Int 6 | 5 | 387.5 | 388.1 |
| 17 | 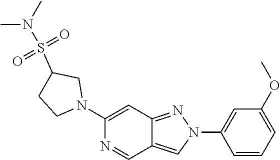 [SMILES: COc1cccc(-n2cc3cnc(N4CCC(S(=O)(=O)N(C)C)C4)cc3n2)c1] | 1-(2-(3-methoxy- phenyl)-2H-pyrazolo [4,3-c]pyridin-6-yl)- N,N-dimethylpyr- rolidine-3-sulfon- amide | Int 23 & Int 6 | 5 | 401.5 | 402.2 |
| 20 | 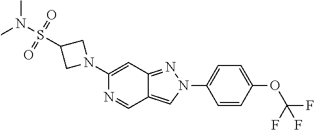 [SMILES: CN(C)S(=O)(=O)C1CN(c2cc3nn(-c4ccc(OC(F)(F)F)cc4)cc3cn2)C1] | N,N-dimethyl-1-(2- (4-(trifluorometh- oxy)phenyl)-2H- pyrazolo[4,3-c]pyr- idin-6-yl)azetidine- 3-sulfonamide | Int 21 & Int 8 | 5 | 441.4 | 442.1 |
| 21 | 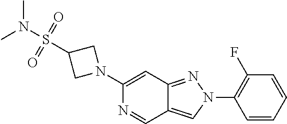 [SMILES: CN(C)S(=O)(=O)C1CN(c2cc3nn(-c4ccccc4F)cc3cn2)C1] | 1-(2-(2-fluorophen- yl)-2H-pyrazolo[4,3- c]pyridin-6-yl)-N,N- dimethylazetidine-3- sulfonamide | Int 21 & Int 15 | 5 | 375.4 | 376.1 |
| 22 | 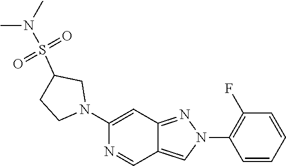 [SMILES: CN(C)S(=O)(=O)C1CCN(c2cc3nn(-c4ccccc4F)cc3cn2)C1] | 1-(2-(2-fluorophen- yl)-2H-pyrazolo[4,3- c]pyridin-6-yl)-N,N- dimethylpyrrolidine- 3-sulfonamide | Int 23 & Int 15 | 5 | 389.5 | 390.1 |
| 24 | 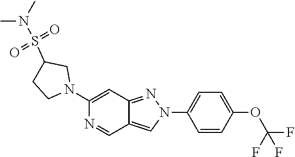 [SMILES: CN(C)S(=O)(=O)C1CCN(c2cc3nn(-c4ccc(OC(F)(F)F)cc4)cc3cn2)C1] | N,N-dimethyl-1-(2- (4-(trifluorometh- oxy)phenyl)-2H-pyr- azolo[4,3-c]pyridin- 6-yl)pyrrolidine-3- sulfonamide | Int 23 & Int 8 | 5 | 455.5 | 456.1 |
| 25 | 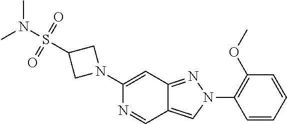 [SMILES: COc1ccccc1-n1cc2cnc(N3CC(S(=O)(=O)N(C)C)C3)cc2n1] | 1-(2-(2-methoxy- phenyl)-2H-pyrazolo [4,3-c]pyridin-6-yl)- N,N-dimethylazeti- dine-3-sulfonamide | Int 21 & Int 6 | 5 | 387.5 | 388.6 |
| 26 | 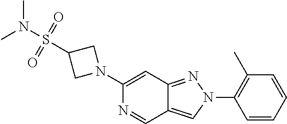 [SMILES: Cc1ccccc1-n1cc2cnc(N3CC(S(=O)(=O)N(C)C)C3)cc2n1] | N,N-dimethyl-1-(2- (o-tolyl)-2H-pyr- azolo[4,3-c]pyridin- 6-yl)azetidine-3- sulfonamide | Int 21 & Int 13 | 5 | 371.5 | 372.6 |
| 27 | 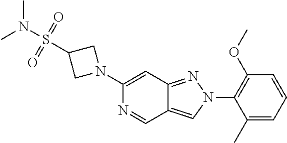 [SMILES: COc1cccc(C)c1-n1cc2cnc(N3CC(S(=O)(=O)N(C)C)C3)cc2n1] | 1-(2-(2-methoxy-6- methylphenyl)-2H- pyrazolo[4,3-c]pyr- idin-6-yl)-N,N- dimethylazetidine- 3-sulfonamide | Int 21 & Int 12 | 5 | 401.5 | 402.6 |
| 28 | 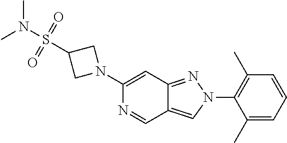 [SMILES: Cc1cccc(C)c1-n1cc2cnc(N3CC(S(=O)(=O)N(C)C)C3)cc2n1] | 1-(2-(2,6-dimethyl- phenyl)-2H-pyr- azolo[4,3-c]pyridin- 6-yl)-N,N-dimethyl- azetidine-3-sulfon- amide | Int 21 & Int 11 | 5 | 385.5 | 386.6 |
| 29 | 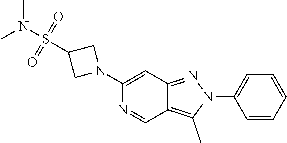 [SMILES: Cc1c2cnc(N3CC(S(=O)(=O)N(C)C)C3)cc2nn1-c1ccccc1] | N,N-dimethyl-1-(3- methyl-2-phenyl- 2H-pyrazolo[4,3-c] pyridin-6-yl)azeti- dine-3-sulfonamide | Int 21 & Int 17 | 5 | 371.5 | 372.1 |
| 30 | 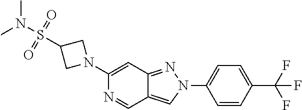 [SMILES: CN(C)S(=O)(=O)C1CN(c2cc3nn(-c4ccc(C(F)(F)F)cc4)cc3cn2)C1] | N,N-dimethyl-1-(2- (4-(trifluoromethyl)- phenyl)-2H-pyrazolo [4,3-c]pyridin-6-yl)- azetidine-3-sulfon- amide | Int 21 & Int 9 | 5 | 425.4 | 426.1 |
| 31 | 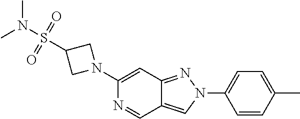 | N,N-dimethyl-1-(2- (p-tolyl)-2H-pyrazolo [4,3-c]pyridin-6-yl)- azetidine-3-sulfon- amide | Int 21 & Int 10 | 5 | 371.5 | 372.1 |
| 32 | 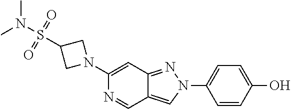 [SMILES: CN(C)S(=O)(=O)C1CN(c2cc3nn(-c4ccc(O)cc4)cc3cn2)C1] | 1-(2-(4-hydroxyphen- yl)-2H-pyrazolo[4,3- c]pyridin-6-yl)-N,N- dimethylazetidine-3- sulfonamide | Int 21 & Int 7 | 5 | 373.4 | 374.1 |
| 37 | 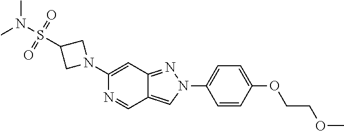 | 1-(2-(4-(2-ethoxy- ethoxy)phenyl)-2H- pyrazolo[4,3-c]pyr- idin-6-yl)-N,N- dimethylazetidine- 3-sulfonamide | Int 21 & Int 16 | 5 | 431.5 | 432.2 |
| 40 | 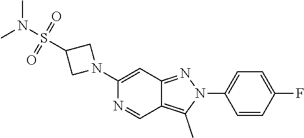 [SMILES: Cc1c2cnc(N3CC(S(=O)(=O)N(C)C)C3)cc2nn1-c1ccc(F)cc1] | 1-(2-(4-fluorophen- yl)-3-methyl-2H- pyrazolo[4,3-c]pyr- idin-6-yl)-N,N- dimethylazetidine- 3-sulfonamide | Int 21 & Int 20 | 5 | 389.5 | 390.2 |
| 41 | 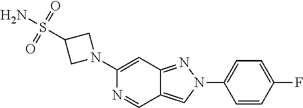 [SMILES: NS(=O)(=O)C1CN(c2cc3nn(-c4ccc(F)cc4)cc3cn2)C1] | 1-(2-(4-fluorophen- yl)-2H-pyrazolo[4,3- c]pyridin-6-yl)azeti- dine-3-sulfonamide | Int 31 & Int 14 | 4-2 | 347.4 | 348.2 |
| 42 | 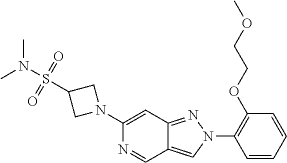 [SMILES: COCCOc1ccccc1-n1cc2cnc(N3CC(S(=O)(=O)N(C)C)C3)cc2n1] | 1-(2-(2-(2-methoxy- ethoxy)phenyl)-2H- pyrazolo[4,3-c]pyr- idin-6-yl)-N,N- dimethylazetidine- 3-sulfonamide | Int 21 & Int 19 | 5 | 431.5 | 432.2 |
| 45 | 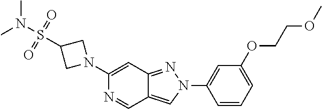 | 1-(2-(3-(2-methoxy- ethoxy)phenyl)-2H- pyrazolo[4,3-c]pyr- idin-6-yl)-N,N- dimethylazetidine- 3-sulfonamide | Int 21 & Int 18 | 5 | 431.5 | 432.2 |
| 54 | 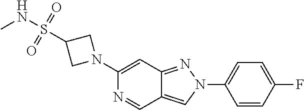 [SMILES: CNS(=O)(=O)C1CN(c2cc3nn(-c4ccc(F)cc4)cc3cn2)C1] | 1-(2-(4-fluorophen- yl)-2H-pyrazolo[4,3- c]pyridin-6-yl)-N- methylazetidine-3- sulfonamide | Int 38 | 4-2 | 361.4 | 362.2 |
| 56 | 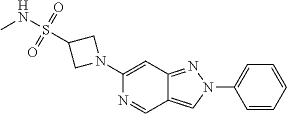 [SMILES: CNS(=O)(=O)C1CN(c2cc3nn(-c4ccccc4)cc3cn2)C1] | N-methyl-1-(2-phen- yl-2H-pyrazolo[4,3- c]pyridin-6-yl)azeti- dine-3-sulfonamide | Int 37 | 4-2 | 343.4 | 344.1 |
| 57 | 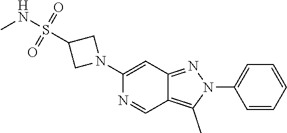 [SMILES: CNS(=O)(=O)C1CN(c2cc3nn(-c4ccccc4)c(C)c3cn2)C1] | N-methyl-1-(3-meth- yl-2-phenyl-2H-pyr- azolo[4,3-c]pyridin- 6-yl)azetidine-3- sulfonamide | Int 53 | 4-2 | 357.4 | 358.1 |
| 58 | 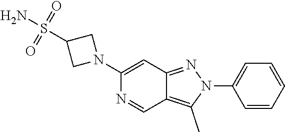 [SMILES: Cc1c2cnc(N3CC(S(N)(=O)=O)C3)cc2nn1-c1ccccc1] | 1-(3-methyl-2-phen- yl-2H-pyrazolo[4,3- c]pyridin-6-yl)azeti- dine-3-sulfonamide | Int 55 | 4-2 | 343.4 | 344.1 |
| 59 | 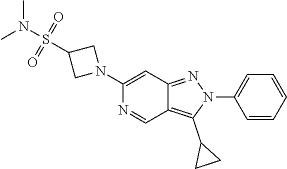 [SMILES: CN(C)S(=O)(=O)C1CN(c2cc3nn(-c4ccccc4)c(C4CC4)c3cn2)C1] | 1-(3-cyclopropyl-2- phenyl-2H-pyrazolo [4,3-c]pyridin-6-yl)- N,N-dimethylazeti- dine-3-sulfonamide | Int 21 & Int 43 | 5 | 397.5 | 398.1 |
| 60 | 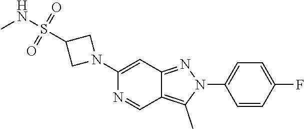 [SMILES: CNS(=O)(=O)C1CN(c2cc3nn(-c4ccc(F)cc4)c(C)c3cn2)C1] | 1-(2-(4-fluorophen- yl)-3-methyl-2H- pyrazolo[4,3-c]pyr- idin-6-yl)-N-methyl- azetidine-3-sulfon- amide | Int 54 | 4-2 | 375.4 | 376.3 |
| 61 | 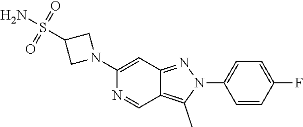 [SMILES: Cc1c2cnc(N3CC(S(N)(=O)=O)C3)cc2nn1-c1ccc(F)cc1] | 1-(2-(4-fluorophen- yl)-3-methyl-2H- pyrazolo[4,3-c]pyr- idin-6-yl)azetidine- 3-sulfonamide | Int 56 | 4-2 | 361.4 | 362.0 |
| 62 | 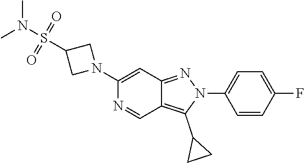 [SMILES: CN(C)S(=O)(=O)C1CN(c2cc3nn(-c4ccc(F)cc4)c(C4CC4)c3cn2)C1] | 1-(3-cyclopropyl-2- (4-fluorophenyl)- 2H-pyrazolo[4,3-c] pyridin-6-yl)-N,N- dimethylazetidine- 3-sulfonamide | Int 21 & Int 44 | 5 | 415.5 | 416.1 |
| 63 | 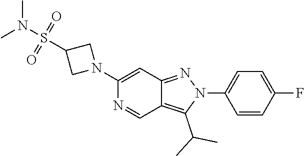 [SMILES: CC(C)c1c2cnc(N3CC(S(=O)(=O)N(C)C)C3)cc2nn1-c1ccc(F)cc1] | 1-(2-(4-fluorophen- yl)-3-isopropyl-2H- pyrazolo[4,3-c]pyr- idin-6-yl)-N,N- dimethylazetidine- 3-sulfonamide | Int 21 & Int 45 | 5 | 417.5 | 418.1 |
| 64 | 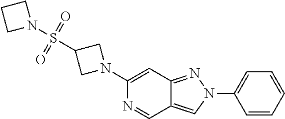 [SMILES: O=S(=O)(C1CN(c2cc3nn(-c4ccccc4)cc3cn2)C1)N1CCC1] | 6-(3-(azetidin-1-yl- sulfonyl)azetidin-1- yl)-2-phenyl-2H-pyr- azolo[4,3-c]pyridine | Int 39 & Int 3 | 5 | 369.4 | 370.1 |
| 65 | 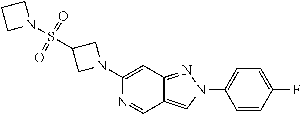 [SMILES: O=S(=O)(C1CN(c2cc3nn(-c4ccc(F)cc4)cc3cn2)C1)N1CCC1] | 6-(3-(azetidin-1-yl- sulfonyl)azetidin-1- yl)-2-(4-fluorophen- yl)-2H-pyrazolo[4,3- c]pyridine | Int 39 & Int 14 | 2-1 | 387.4 | 388.1 |
| 66 | 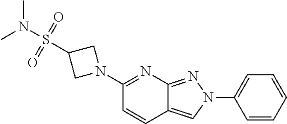 [SMILES: CN(C)S(=O)(=O)C1CN(c2ccc3cn(-c4ccccc4)nc3n2)C1] | N,N-dimethyl-1-(2- phenyl-2H-pyrazolo [3,4-b]pyridin-6-yl)- azetidine-3-sulfon- amide | Int 48 & CAS# 62-53-3 | 2-1 | 357.4 | 358.1 |
| 67 | 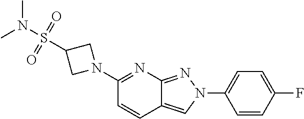 [SMILES: CN(C)S(=O)(=O)C1CN(c2ccc3cn(-c4ccc(F)cc4)nc3n2)C1] | 1-(2-(4-fluorophen- yl)-2H-pyrazolo[3,4- b]pyridin-6-yl)-N,N- dimethylazetidine-3- sulfonamide | Int 48 & CAS# 371-40-4 | 2-1 | 375.4 | 376.2 |
| 68 | 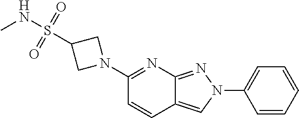 [SMILES: CNS(=O)(=O)C1CN(c2ccc3cn(-c4ccccc4)nc3n2)C1] | N-methyl-1-(2-phen- yl-2H-pyrazolo[3,4- b]pyridin-6-yl)azeti- dine-3-sulfonamide | Int 52 | 4-2 | 343.4 | 344.7 |
| 69 | 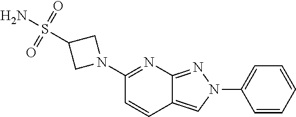 [SMILES: NS(=O)(=O)C1CN(c2ccc3cn(-c4ccccc4)nc3n2)C1] | 1-(2-phenyl-2H-pyr- azolo[3,4-b]pyridin- 6-yl)azetidine-3- sulfonamide | Int 51 | 4-2 | 329.4 | 330.0 |

TABLE IV
NMR data of illustrative compounds of the invention and comparative compounds.

| Cpd# | NMR |
|---|---|
| 1 | ¹H NMR (400 MHz, DMSO-d₆) δ = 9.23 (s, 1H), 8.98 (d, 1H), 8.05 (d, 2H), 7.58 (t, 2H), 7.45 |
| | (t, 1H), 6.38 (s, 1H), 4.56-4.49 (m, 1H), 4.28 (t, 2H), 4.13 (dd, 2H), 2.81 (s, 6H) |
| 2 | ¹H NMR (400 MHz, DMSO-d₆) δ = 9.19 (s, 1H), 8.98 (d, 1H), 8.05 (d, 2H), 7.57 (t, 2H), 7.46- |
| | 7.39 (m, 1H), 6.33 (s, 1H), 4.27-4.15 (m, 1H), 3.88-3.80 (m, 1H), 3.75-3.66 (m, 1H), |
| | 3.65-3.57 (m, 1H), 3.48-3.37 (m, 1H), 2.86 (s, 6H), 2.70-2.51 (m, 2H) |
| 3 | ¹H NMR (400 MHz, DMSO-d₆) δ = 9.20 (s, 1H), 9.01 (d, 1H), 8.08-8.02 (m, 2H), 7.63- |
| | 7.53 (m, 2H), 7.51-7.40 (m, 1H), 6.67 (s, 1H), 4.83-4.71 (m, 1H), 4.18-4.05 (m, 1H), 3.44- |
| | 3.32 (m, 1H), 2.98-2.90 (m, 1H), 2.88 (s, 6H), 2.85-2.80 (m, 1H), 2.15-2.05 (m, 1H), |
| | 1.83-1.56 (m, 3H) |
| 5 | ¹H NMR (400 MHz, DMSO-d₆) δ = 9.23 (s, 1H), 8.98 (s, 1H), 8.05 (d, 2H), 7.63-7.53 (m, |
| | 2H), 7.50-7.40 (m, 1H), 6.37 (s, 1H), 4.54-4.44 (m, 1H), 4.23 (t, 2H), 4.13-4.01 (m, 3H), |
| | 2.72 (s, 3H), 1.11 (d, 6H) |
| 7 | ¹H NMR (400 MHz, DMSO-d₆) δ = 9.24 (s, 1H), 8.99 (s, 1H), 8.06 (d, 2H), 7.59 (t, 2H), 7.52- |
| | 7.40 (m, 1H), 6.39 (s, 1H), 4.68-4.60 (m, 1H), 4.33 (t, 2H), 4.18 (t, 2H), 2.84 (s, 3H), 2.46- |
| | 2.41 (m, 1H), 0.74 (s, 4H) |
| 8 | ¹H NMR (400 MHz, DMSO-d₆) δ = 9.21 (s, 1H), 8.98 (s, 1H), 8.04 (d, 2H), 7.58 (t, 2H), 7.48- |
| | 7.40 (m, 1H), 7.14 (s, 2H), 6.31 (s, 1H), 4.25-4.18 (m, 3H), 4.15-4.08 (m, 2H) |
| 9 | ¹H NMR (400 MHz, DMSO-d₆) δ = 9.13 (s, 1H), 8.95 (s, 1H), 7.97 (d, 2H), 7.13 (d, 2H), 6.37 |
| | (s, 1H), 4.58-4.51 (m, 1H), 4.28 (t, 2H), 4.15-4.10 (m, 2H), 3.83 (s, 3H), 2.82 (s, 6H) |
| 10 | ¹H NMR (400 MHz, DMSO-d₆) δ = 9.08 (s, 1H), 8.95 (s, 1H), 7.97 (d, 2H), 7.12 (d, 2H), 6.33 |
| | (s, 1H), 4.24-4.18 (m, 1H), 3.85-3.81 (m, 4H), 3.69 (dd, 1H), 3.64-3.60 (m, 1H), 3.48- |
| | 3.39 (m, 1H), 2.87 (s, 6H), 2.44-2.37 (m, 1H), 2.34-2.26 (m, 1H) |
| 14 | ¹H NMR (400 MHz, DMSO-d₆) δ = 9.22 (s, 1H), 9.00 (s, 1H), 8.13-8.09 (m, 2H), 7.47-7.43 |
| | (m, 2H), 6.39 (s, 1H), 4.58-4.53 (m, 1H), 4.32-4.27 (m, 2H), 4.16-4.12 (m, 2H), 2.83 (s, |
| | 6H) |
| 15 | ¹H NMR (400 MHz, DMSO-d₆) δ = 9.13 (s, 1H), 8.97 (s, 1H), 8.07 (dd, 2H), 7.42 (t, 2H), |
| | 6.30 (s, 1H), 4.24-4.13 (m, 1H), 3.89-3.75 (m, 1H), 3.68 (dd, 1H), 3.64-3.56 (m, 2H), 2.85 |
| | (s, 6H), 2.46-2.27 (m, 2H) |
| 16 | ¹H NMR (400 MHz, DMSO-d₆) δ = 9.27 (s, 1H), 8.98 (s, 1H), 7.63-7.65 (m, 2H), 7.49 (t, |
| | 1H), 7.03 (d, 1H), 6.38 (s, 1H), 4.59-4.51 (m, 1H), 4.29 (t, 2H), 4.13 (dd, 2H), 3.87 (s, 3H), |
| | 2.82 (s, 6H) |
| 17 | ¹H NMR (400 MHz, DMSO-d₆) δ = 9.22 (s, 1H), 8.98 (s, 1H), 7.62-7.65 (m, 2H), 7.48 (t, |
| | 1H), 7.02-6.99 (m, 1H), 6.33 (s, 1H), 4.25-4.17 (m, 1H), 3.87 (s, 3H), 3.85-3.82 (m, 1H), |
| | 3.70 (dd, 1H), 3.67-3.60 (m, 1H), 3.49-3.38 (m, 1H), 2.87 (s, 6H), 2.44-2.37 (m, 1H), 2.35- |
| | 2.28 (m, 1H) |
| 20 | ¹H NMR (400 MHz, DMSO-d₆) δ = 9.27 (d, 1H), 9.00 (d, 1H), 8.21-8.18 (m, 2H), 7.61- |
| | 7.59 (m, 2H), 6.37 (s, 1H), 4.57-4.51 (m, 1H), 4.28 (t, 2H), 4.13 (dd, 2H), 2.81 (s, 6H) |
| 21 | ¹H NMR (400 MHz, DMSO-d₆) δ = 9.01 (d, 1H), 8.98 (dd, 1H), 8.00-7. 90 (m, 1H), 7.61- |
| | 7.49 (m, 2H), 7.49-7.38 (m, 1H), 6.37 (s, 1H), 4.64-4.43 (m, 1H), 4.28 (t, 2H), 4.17-4.09 |
| | (m, 2H), 2.81 (s, 6H) |
| 22 | ¹H NMR (400 MHz, DMSO-d₆) δ = 9.01 (d, 1H), 8.94-8.91 (m, 1H), 8.00-7.93 (m, 1H), |
| | 7.58-7.50 (m, 2H), 7.48-7.37 (m, 1H), 6.31 (s, 1H), 4.25-4.15 (m, 1H), 3.89-3.78 (m, |
| | 1H), 3.74-3.66 (m, 1H), 3.65-3.58 (m, 1H), 3.51-3.38 (m, 1H), 2.86 (s, 6H), 2.42-2.28 |
| | (m, 2H) |
| 24 | ¹H NMR (400 MHz, DMSO-d₆) 8) = 9.22 (d, 1H), 9.00 (d, 1H), 8.15-8.20 (m, 2H), 7.59 (d, |
| | 2H), 6.32 (s, 1H), 4.24-4.16 (m, 1H), 3.84 (dd, 1H), 3.69 (dd, 1H), 3.66-3.59 (m, 1H), 3.47- |
| | 3.39 (m, 1H), 2.86 (s, 6H), 2.47-2.27 (m, 2H) |
| 25 | ¹H NMR (400 MHz, DMSO-d₆) δ = 8.98 (d, 1H), 8.93 (s, 1H), 7.79 (dd, 1H), 7.51-7.46 (m, |
| | 1H), 7.33 (d, 1H), 7.15 (t, 1H), 6.35 (s, 1H), 4.58-4.52 (m, 1H), 4.28 (t, 2H), 4.13 (dd, 2H), |
| | 3.90 (s, 3H), 2.82 (s, 6H) |
| 26 | ¹H NMR (400 MHz, DMSO-d₆) δ = 8.99 (s, 1H), 8.78 (s, 1H), 7.57-7.34 (m, 4H), 6.39 (s, |
| | 1H), 4.61-4.47 (m, 1H), 4.28 (t, 2H), 4.20-4.06 (m, 2H), 2.82 (s, 6H), 2.19 (s, 3H) |
| 27 | ¹H NMR (400 MHz, DMSO-d₆) δ = 8.96 (d, 1H), 8.61 (s, 1H), 7.44 (t, 1H), 7.10 (d, 1H), 7.01 |
| | (d, 1H), 6.36 (s, 1H), 4.58-4.51 (m, 1H), 4.28 (t, 2H), 4.12 (dd, 2H), 3.70 (s, 3H), 2.82 (s, |
| | 6H), 1.94 (s, 3H) |
| 28 | ¹H NMR (400 MHz, DMSO-d₆) δ = 8.99 (s, 1H), 8.70 (s, 1H), 7.38 (t, 1H), 7.27 (d, 2H), 6.40 |
| | (s, 1H), 4.59-4.52 (m, 1H), 4.29 (t, 2H), 4.13 (dd, 2H), 2.82 (s, 6H), 1.92 (s, 6H) |
| 29 | ¹H NMR (400 MHz, DMSO-d₆) δ = 8.98 (s, 1H), 7.69-7.65 (m, 2H), 7.60 (t, 2H), 7.57-7.53 |
| | (m, 1H), 6.26 (s, 1H), 4.57-4.51 (m, 1H), 4.27 (t, 2H), 4.11 (dd, 2H), 2.82 (s, 6H), 2.68 (s, |
| | 3H) |
| 30 | ¹H NMR (400 MHz, DMSO-d₆) δ = 9.40 (s, 1H), 9.05-9.04 (m, 1H), 8.31 (d, 2H), 7.97 (d, |
| | 2H), 6.38 (s, 1H), 4.60-4.56 (s, 1H), 4.30 (t, 2H), 4.15 (dd, 2H), 2.82 (s, 6H) |
| 31 | ¹H NMR (400 MHz, DMSO-d₆) δ = 9.19 (s, 1H), 8.97 (s, 1H), 7.94 (d, 2H), 7.39 (d, 2H), 6.37 |
| | (s, 1H), 4.57-4.54 (m, 1H), 4.28 (t, 2H), 4.16-4.10 (m, 2H), 2.82 (s, 6H), 2.38 (s, 3H) |
| 32 | ¹H NMR (400 MHz, DMSO-d₆) δ = 9.91 (s, 1H), 9.04 (s, 1H), 8.94-8.92 (m, 1H), 7.83 (d, |
| | 2H), 6.93 (d, 2H), 6.36 (s, 1H), 4.56-4.51 (m, 1H), 4.27 (t, 2H), 4.12 (dd, 2H), 2.82 (s, 6H) |
| 37 | ¹H NMR (400 MHz, DMSO-d₆) δ = 9.12 (s, 1H), 8.94 (s, 1H), 7.98-7.92 (m, 2H), 7.15-7.10 |
| | (m, 2H), 6.36 (s, 1H), 4.55-4.50 (m, 1H), 4.27 (t, 2H), 4.19-4.14 (m, 2H), 4.12 (dd, 2H), |
| | 3.71-3.65 (m, 2H), 2.81 (s, 6H) |
| 40 | ¹H NMR (400 MHz, DMSO-d₆) δ = 8.97 (s, 1H), 7.73 (dd, 2H), 7.44 (t, 2H), 6.25 (s, 1H), |
| | 4.58-4.50 (m, 1H), 4.27 (t, 2H), 4.11 (dd, 2H), 2.82 (s, 6H), 2.66 (s, 3H) |
| 41 | ¹H NMR (400 MHz, DMSO-d₆) δ = 9.19 (d, 1H), 8.98 (d, 1H), 8.12-8.05 (m, 2H), 7.47- |
| | 7.39 (m, 2H), 7.14 (s, 2H), 6.32-6.29 (m, 1H), 4.26-4.16 (m, 3H), 4.15-4.08 (m, 2H) |
| 42 | ¹H NMR (400 MHz, DMSO-d₆) δ = 8.98 (d, 2H), 7.85 (dd, 1H), 7.45-7.39 (m, 1H), 7.37- |
| | 7.31 (m, 1H), 7.19-7.09 (m, 1H), 6.35 (s, 1H), 4.60-4.47 (m, 1H), 4.31-4.24 (m, 4H), 4.12 |
| | (dd, 2H), 3.72-3.65 (m, 2H), 3.28 (s, 3H), 2.81 (s, 6H) |
| 45 | ¹H NMR (400 MHz, DMSO-d₆) δ = 9.26 (d, 1H), 8.97 (d, 1H), 7.65-7.61 (m, 2H), 7.47 (t, |
| | 1H), 7.04-6.99 (m, 1H), 6.37 (s, 1H), 4.60-4.49 (m, 1H), 4.28 (t, 2H), 4.23-4.17 (m, 2H), |
| | 4.12 (dd, 2H), 3.72-3.67 (m, 2H), 3.32 (s, 3H), 2.81 (s, 6H) |
| 54 | ¹H NMR (400 MHz, CD₃OD) δ = 9.21 (s, 1H), 9.00-8.97 (m, 1H), 8.13-8.07 (m, 2H), 7.47- |
| | 7.41 (m, 2H), 7.25 (s, 1H), 6.35 (s, 1H), 4.44-4.38 (m, 1H), 4.23 (t, 2H), 4.13-4.07 (m, |
| | 2H), 2.64 (s, 3H) |
| 56 | ¹H NMR (400 MHz, DMSO-d₆) δ = 9.24 (s, 1H), 9.00-8.98 (m, 1H), 8.06 (d, 2H), 7.59 (t, |
| | 2H), 7.46 (t, 1H), 7.24 (d, 1H), 6.36 (s, 1H), 4.45-4.38 (m, 1H), 4.24 (t, 2H), 4.10 (dd, 2H), |
| | 2.64 (d, 3H) |
| 57 | ¹H NMR (400 MHz, DMSO-d₆) δ = 8.96 (d, 1H), 7.68-7.63 (m, 2H), 7.63-7.50 (m, 3H), |
| | 7.26-7.18 (m, 1H), 6.22 (d, 1H), 4.45-4.34 (m, 1H), 4.20 (t, 2H), 4.11-4.04 (m, 2H), 2.67 |
| | (s, 3H), 2.65-2.61 (m, 3H) |
| 58 | ¹H NMR (400 MHz, DMSO-d₆) δ = 9.02 (s, 1H), 7.69-7.64 (m, 2H), 7.64-7.52 (m, 3H), |
| | 7.15 (s, 2H), 6.28-6.23 (m, 1H), 4.26-4.17 (m, 3H), 4.16-4.08 (m, 2H), 2.68 (s, 3H) |
| 59 | ¹H NMR (400 MHz, DMSO-d₆) δ = 8.88 (d, 1H), 7.74-7.68 (m, 2H), 7.63-7.52 (m, 3H), |
| | 6.28-6.25 (m, 1H), 4.58-4.47 (m, 1H), 4.25 (t, 2H), 4.09 (dd, 2H), 2.80 (s, 6H), 2.26-2.18 |
| | (m, 1H), 1.10-0.94 (m, 4H) |
| 60 | ¹H NMR (400 MHz, DMSO-d₆) δ = 8.97 (s, 1H), 7.76-7.70 (m, 2H), 7.44 (t, 2H), 7.23 (s, |
| | 1H), 6.22 (s, 1H), 4.43-4.37 (m, 1H), 4.21 (t, 2H), 4.08 (dd, 2H), 2.66 (s, 3H), 2.64 (s, 3H) |
| 61 | ¹H NMR (400 MHz, DMSO-d₆) δ = 8.96 (d, 1H), 7.76-7.67 (m, 2H), 7.47-7.39 (m, 2H), |
| | 7.13 (s, 2H), 6.18 (d, 1H), 4.23-4.16 (m, 3H), 4.14-4.07 (m, 2H), 2.65 (s, 3H) |
| 62 | ¹H NMR (400 MHz, DMSO-d₆) δ = 8.89 (d, 1H), 7.81-7.74 (m, 2H), 7.48-7.40 (m, 2H), |
| | 6.26 (s, 1H), 4.57-4.48 (m, 1H), 4.25 (t, 2H), 4.09 (dd, 2H), 2.80 (s, 6H), 2.25-2.16 (m, |
| | 1H), 1.11-1.00 (m, 2H), 1.00-0.92 (m, 2H) |
| 63 | ¹H NMR (400 MHz, DMSO-d₆) δ = 9.15 (s, 1H), 7.62 (dd, 2H), 7.48-7.42 (m, 2H), 6.27 (s, |
| | 1H), 4.57-4.50 (m, 1H), 4.26 (t, 2H), 4.14-4.07 (m, 2H), 3.21-3.15 (m, 1H), 2.81 (s, 6H), |
| | 1.42 (d, 6H) |
| 64 | ¹H NMR (400 MHz, DMSO-d₆) δ = 9.24 (d, 1H), 8.99 (d, 1H), 8.07-8.05 (m, 2H), 7.61- |
| | 7.57 (m, 2H), 7.48-7.44 (m, 1H), 6.39 (t, 1H), 4.62-4.56 (m, 1H), 4.30 (t, 2H), 4.09 (dd, |
| | 2H), 3.93-3.90 (m, 4H), 2.24-2.19 (m, 2H) |
| 65 | ¹H NMR (400 MHz, DMSO-d₆) δ = 9.22 (s, 1H), 8.99 (s, 1H), 8.14-8.07 (m, 2H), 7.45 (t, |
| | 2H), 6.38 (s, 1H), 4.62-4.54 (m, 1H), 4.29 (t, 2H), 4.09 (dd, 2H), 3.91 (t, 4H), 2.24-2.17 |
| | (m, 2H) |
| 66 | ¹H NMR (400 MHz, DMSO-d₆) δ = 8.89 (s, 1H), 8.02-7.97 (m, 3H), 7.55 (t, 2H), 7.40-7.36 |
| | (m, 1H), 6.50 (d, 1H), 4.57-4.52 (m, 1H), 4.41 (t, 2H), 4.26 (dd, 2H), 2.84 (s, 6H) |
| 67 | ¹H NMR (400 MHz, DMSO-d₆) δ = 8.85 (s, 1H), 8.06-7.96 (m, 3H), 7.44-7.36 (m, 2H), |
| | 6.49 (d, 1H), 4.59-4.48 (m, 1H), 4.40 (t, 2H), 4.29-4.21 (m, 2H), 2.82 (s, 6H) |
| 68 | ¹H NMR (400 MHz, DMSO-d₆) δ = 8.87 (s, 1H), 8.02-7.94 (m, 3H), 7.59-7.49 (m, 2H), |
| | 7.40-7.35 (m, 1H), 7.31-7.27 (m, 1H), 6.48 (d, 1H), 4.42-4.31 (m, 3H), 4.25-4.15 (m, |
| | 2H), 2.64 (d, 3H) |
| 69 | ¹H NMR (400 MHz, DMSO-d₆) δ = 8.86 (s, 1H), 7.97 (dd, 3H), 7.54 (t, 2H), 7.41-7.34 (m, |
| | 1H), 7.20 (s, 2H), 6.46 (d, 1H), 4.40-4.28 (m, 2H), 4.27-4.19 (m, 3H) |

### Biological Examples

### Example 2. In Vitro Assays

### 2.1. MDCKII-MDR1

### 2.1.1. Study Principle

The aim of this study is determine the permeability and the efflux ratio of the compounds of the invention in Madin-Darby Canine Kidney (MDCKII) cells.

### 2.1.2. Study Protocol

MDCKII-MDR1 cells are seeded on Millicell-24 cell culture insert plate assemblies, at a final concentration of 0.12×106 cells/well. Cells are cultured in a CO2 incubator for 3-4 days prior to experiment start with media replacement 24 hours post seeding.

On the day of the experiment, cells are pre-incubated for 45 minutes with Dulbecco's Phosphate Buffer saline (D-PBS, pH7.4), containing 1% of DMSO. Compounds are prepared in D-PBS, pH7.4 and added to either the apical or basolateral chambers of the Millicell cell culture insert plates assembly at a final concentration of 10 μM with a final DMSO concentration of 1%. Lucifer Yellow is added to all donor buffer solutions, in order to assess integrity of the cell monolayers by monitoring Lucifer Yellow permeation. After a 1 hour of incubation at 37° C., while shaking, aliquots are taken from both apical (A) and basolateral (B) chambers and added to ACN:water solution (2:1) containing analytical internal standard.

Samples are also taken at the beginning of the experiment from donor solutions to obtain initial (CO) concentration. After brief mixing and centrifugation, the supernatant is analyzed by LC-MS/MS. The apparent permeability coefficient (Papp) is calculated according to the following equation: Papp=(dQ/dT)*(1/CO)*(1/A) where dQ/dT=permeability rate; CO=initial concentration in donor compartment; A=surface area of the cell monolayer (0.7cm2). “Concentration” is the ratio between compound and internal standard peak areas. The Papp value has a dimension of a rate (x10-6 cm/sec). The efflux ratio is calculated as Papp from B to A divided by Papp from A to B. Passive permeability (x10-6 cm/sec) is calculated by the formula: Papp from A to B×(efflux ratio+1/2).

### Example 3. In vivo assays

### 3.1. Alzheimer's Disease Mouse Model Using Intrahippocampal Injection of Oligomers of Amyloid Beta.

### 3.1.1. Study Principle

Increased levels of amyloid f (Ap) can be found in the brain of the AD patients. This is accompanied by the activation of microglia and an increase in the number of microglia in AD brains, referred to as microgliosis. Activated microglia can be found early in the disease progression.

The aim of this Alzheimer's disease model was to assess effect of a test compound based on intra-hippocampal injections of ApO which have been linked to AD pathogenesis.(Sakono and Zako 2010), and induce short- and long-term memory deficits (Calvo-Flores Guzmin et al. 2020). The cognitive dysfunctions are associated and well correlated with the neuronal loss and the activation of microglial cells.

This study evaluated the cognitive functions of the mice after a chronic administration of the compounds, after the ApO injury. Samples of plasma and brains were also collected for further histological analysis, evaluation via immunochemistry of neurodegeneration and activation of microglial cells.

### 3.1.2. Study Protocol

### 3.1.2.1. Overview

The forced alternation Y-maze test relies on the natural tendency of rodents to explore new environments and was used to evaluate short-term spatial memory and exploratory behavior of injured mice.

### 3.1.2.2. Animals

18-month-old male C57BL6 mice (Janvier Labs) were housed for acclimatation for 1 week in Neuro-Sys facilities and were maintained in a reversed 12 h light-dark cycle. The animals were group-housed (2-4 animals per cage) and maintained in room with controlled temperature (21-22° C.) and hygrometry (40-60%) and with food and water available ad libitum.

3.1.2.3. Stereotaxic injections of Aβ1-42 preparation

After one week of acclimatization, all the mice were subjected to a bilateral hippocampal stereotaxic surgery determined relative to the bregma according to the Paxinos and Franklin's mouse brain atlas(Yeung et al. 2020).

The Aβ1-42 preparation were done following the procedure described by Callizot (Callizot et al. 2013). Briefly, Aβ1-42 peptide (Bachem, 1071428, CAS #107761-42-2) was dissolved in the vehicle (0.5% Methylcellulose in water (w/v)), at an initial concentration of 100 μM. This solution was gently agitated for 3 days at 37° C. in the dark.

After agitation, the Aβ1-42 preparation were bilaterally injected into the stratum oriens, striatum pyramidal and striatum radiatum of the CA1 area of the hippocampus at three different depths within the CA1 region. Stereotaxic bilateral administration of 2 μL/side of Aβ1-42 preparation (100 μM, containing ˜15 mol/L of oligomers precisely measured by automatic WB), or vehicle, were bilaterally injected with a Hamilton syringe in the CA 1 area of the hippocampus (0.2 μL/min with an Elite Nanomite syringe pump).

### 3.1.2.4. Study Design

All treatments started the day before the stereotaxic injections of A31-42 and were administrated daily until the last day of the experiment at which point different tissue samples were collected.

Test compounds were administered by oral gavage (p.o.).

Donepezil (DNP) is an inhibitor of acetylcholine esterase which acts as a potentiator of acetylcholine signaling is approved by the FDA for the management of mild cognitive impairment. DNP was administered intraperitonally (i.p.)

TABLE V
Study groups and duration

| | | | Sacrifice |
|---|---|---|---|
| | Group | | (days post- |
| Group ID | (n = 12) | Treatment | surgery) |
| 1 | Sham | Vehicle 1 | 18 |
| 2 | Aβ1-42 | Vehicle 1 | 18 |
| | (2 μL/side, 100 μM) | | |
| 3 | Aβ1-42 | Compound 14 | 18 |
| | (2 μL/side, 100 μM) | (5 mg/kg, vehicle 1) | |
| 6 | Aβ1-42 | Compound 66 | 18 |
| | (2 μL/side, 100 μM) | (5 mg/kg, vehicle 2) | |
| 8 | Aβ1-42 | Donepezil | 18 |
| | (2 μL/side, 100 μM) | (1 mg/kg, saline) | |

Preparation with vehicle 1: The required amount of the test compound was weighted and placed in a glass tube. The compound was dissolved in 0.5% Methylcellulose in water (w/v) corresponding to 100% of the final volume and vortexed. The formulation (glass vial) was placed in an ultrasonic bath for 30 min.

The preparation was aliquoted in tubes protected from light and stored at 4° C.

Preparation with vehicle 2: The required amount of the test compound was weighted and placed in a glass tube. The compound was dissolved in a volume PEG200 corresponding to 25% of the final volume (at 4× concentration). The preparation was aliquoted in tubes protected from light and stored at 4° C. Before administration, a volume of MC (0.5%, w/v in sterile water) corresponding to 75% of the final volume (for 1× concentration) was added to the preparation and vortexed. The formulation (glass vial) was placed in an ultrasonic bath for homogenization. The glass vial containing the preparation was kept under agitation and protected from light until administration.

### 3.1.2.5. Evaluation of Short-Term Spatial Memory- Y- Maze

The Y-maze was used to assess short term memory (working memory) in mice. Spatial reference memory, which is underlined by the hippocampus, was tested by placing the test mice into the Y-maze with one arm closed off during training. After an intertrial interval, the mouse should remember which arm it has not explored previously and should visit this arm more often (Kraeuter, Guest, and Samyai 2019). At the back of each arms, some visual cues (sticked on the walls) were put to allow the animals some visual identification.

The apparatus consisted in a light-grey colored polyvinylchloride (PVC) Y-shaped compartment (35 cm arm length×6 cm×15 cm height arm) with equal length arms.

Before the day of the surgery, a training session of Y-maze was performed, as an acclimatization session for all mice. Seven (7) days and fourteen (14) days after the day of the surgery, mice were tested again.

The test was based on two trials:
1. Mice were allowed to freely explore two arms of the Y-maze during 5 min (300 s). The last arm remained closed. At the end of the 5 min, the animals were let for 3 min in an empty cage. The Y-maze was cleaned with acetic acid to neutralize any odor.
2. After the 3 min. the mice were then allowed to freely explore the three arms of the Y-maze during 5 min.

Both trials were automatically recorded by a video camera using Ethovision system (Noldus). The number of entries, and the time spent in each arm were automatically determined for each animal.

The animals staying in the entry arm for the entire test duration (<200 s) were automatically excluded from the analysis.

The animals displaying freezing (by an over-anxiety behavior) were excluded from the test.

### 3.1.2.6. Immunostaining (IHC).

At the end of the experiment (on day 18), mice (n=5) from the groups 1, 2 and 3 were sacrificed under anesthesia, brains were collected and fixed in paraformaldehyde (PFA) 4%, for 2 h at room temperature. After 2 hours, the brains were placed in 30% sucrose in Tris-phosphate saline (TBS) solution overnight at 4° C.

Serial coronal sections, including the hippocampus area, of 40 μm-thickness were cut using a freezing microtome.

For immunostaining, free-floating sections were incubated in TBS with 0.25% bovine serum albumin, 0.3% Triton X-100 and 10% goat serum, for 1 hour at room temperature. This incubation was made to block unspecific binding sites and to permeabilize tissues.

### 3.1.2.7. Bioassays (ELISA and NGS)

At the end of the experiment (on day 18), mice (n=7 maximum from groups 1, 2, 3 and 8) were sacrificed under anesthesia and specific brain areas were collected. The 2 hemispheres were split in order to generate two cortices (one from each hemisphere) and two hippocampal areas (one from each hemisphere) per animal. These samples were kept at −80° C.

### 3.1.2.8. Pharmacokinetic Samples and Brain Sampling for Bioassays (ELISA and NCS)

### 3.1.2.8.1.1. PK Blood Sampling:

At the end of the experiment (on day 8 or day 18), for mice (n=9,3 samples par timepoint) (from group 6), blood was collected under deep anesthesia in EDTA-tubes at 0.5, 1 and 4h after the last treatment for PK analysis. 25 μL of blood were transferred into a second EDTA-tube containing 25 μL of deionized water and kept frozen at −80° C. The rest of the blood was centrifuged and the plasma was collected and kept at −80° C.

### 3.1.2.8.2 Brain Sampling:

The brains from the same animals were dissected. The 2 hemispheres were split in order to generate two cortices (one from each hemisphere) and two hippocampal areas (one from each hemisphere) per animal. These samples were kept at −80° C.

### 3.1.2.9. Statistical Analysis

All values show the mean+/−SEM (standard error of the mean) per group of animals. Graphs and statistical analyses were performed either via one-way or two-way ANOVA followed by Fisher's test; using GraphPad Prism software version 8.0.2 *p<0.05 was considered significant. Outliers were identified by Grubb's test (alpha=0.2) and by abnormal behavior during the Y-Maze test (e.g. freezing behavior during the test).

### 3.1.3. Results

A significant impairment in the short-term spatial memory was found 7 days post-surgery in the Aβ3l-42-injected mice compared to the control animals (Table VI), this was demonstrated by a reduced exploration time of the new arm during the forced alternation test of the Y-maze. Short-term memory defect remained significantly altered in a second Y-maze test, performed 14 days after surgery, when compared to controls.

TABLE VI
Results of the study group

| | | | | | p-value |
|---|---|---|---|---|---|
| | | | Cumulative | Standard | (one-way |
| Group ID | Group (n = 12) | Treatment | duration (sec) | Deviation | ANOVA) |
| 1 | Sham | Vehicle 1 | 115.5 | 22.8 | 0.0208 |
| 2 | Aβ1-42 | Vehicle 1 | 78.9 | 23.4 | NA |
| 3 | (2 μL/side, | Compound 14 | 112.5 | 21.6 | 0.0102 |
| | 100 μM) | (5 mg/kg, | | | |
| | | vehicle 1) | | | |
| 6 | Aβ1-42 | Compound 66 | 104.4 | 33.8 | 0.0484 |
| | | (5 mg/kg, | | | |
| | | vehicle 2) | | | |
| 8 | (2 μL/side, | Donepezil | 134.6 | 44.3 | 0.0009 |
| | 100 μM) | (1 mg/kg, saline) | | | |

NA = Not applicable

Compound 14 showed significant beneficial effects on short-term spatial memory on day 7 and day 14 post-surgery when compared to the Ap 1-42- injected mice and vehicle treated (group ID 2).

Compound 66 showed significant beneficial effects on short-term spatial memory on day 7 when compared to group ID 2.

### DESCRIPTION OF FIGURES

FIG. 1 describes the average cumulative duration (in seconds) of each mice group in the new arm of the Y-maze. Each column of the plot represents the average time together with the standard deviation ranges of each mice group described in Table V. Column A represents the control group (group ID 1), column B represents the group ID 2, column C represents the group ID 3, column D represents the group ID 6 and column represents the group ID 8.

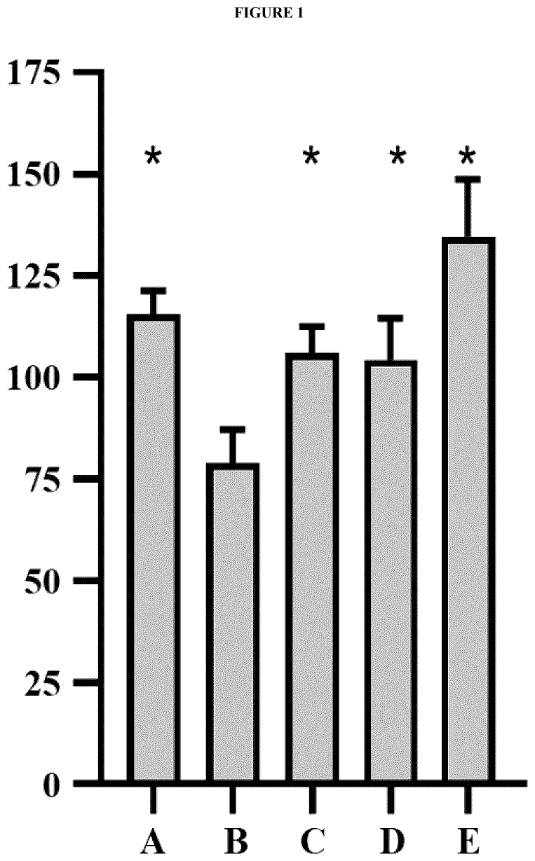

The * symbol on top of a column represents that the p-value is <0.05 versus the group ID 2 (one-way ANOVA followed by the Fisher's test).

### Final Remarks

It will be appreciated by those skilled in the art that the foregoing descriptions are exemplary and explanatory in nature and intended to illustrate the invention and its preferred embodiments. Through routine experimentation, an artisan will recognize apparent modifications and variations that may be made without departing from the spirit of the invention. All such modifications coming within the scope of the appended claims are intended to be included therein. Thus, the invention is intended to be defined not by the above description, but by the following claims and their equivalents.

All publications, including but not limited to patents and patent applications, cited in this specification are herein incorporated by reference as if each individual publication are specifically and individually indicated to be incorporated by reference herein as though fully set forth.

It should be understood that factors such as the differential cell penetration capacity of the various compounds can contribute to discrepancies between the activity of the compounds in the in vitro biochemical and cellular assays.

At least some of the chemical names of compound of the invention as given and set forth in this application, may have been generated on an automated basis by use of a commercially available chemical naming software program, and have not been independently verified. Representative programs performing this function include the Lexichem naming tool sold by Open Eye Software, Inc. and the Autonom Software tool sold by MDL, Inc. In the instance where the indicated chemical name and the depicted structure differ, the depicted structure will control.

### REFERENCES

Bundgaard, Hans. 1985. Design of Prodrugs. Amsterdam; New York; Oxford: Elsevier.
Callizot, Noelle, Maud Combes, R⁶my Steinschneider, and Philippe Poindron. 2013. “Operational Dissection of D-Amyloid Cytopathic Effects on Cultured Neurons.” Journal of Neuroscience Research 91 (5): 706-16. https://doi.org/10.1002/jnr.23193.
Calvo-Flores Guzmán, Beatriz, SooHyun Kim, Bhavya Chawdhary, Katie Peppercorn, Warren P Tate, Henry J Waldvogel, Richard LM Faull, Johanna Montgomery, and Andrea Kwakowsky. 2020. “Amyloid-Betal-42-Induced Increase in GABAergic Tonic Conductance in Mouse Hippocampal CA1 Pyramidal Cells.” Molecules 25 (3): 693. https://doi.org/10.3390/molecules25030693.
“Greene's Protective Groups in Organic Synthesis, 4th Edition | Wiley.” 2006. Wiley.Com. 2006. https://www.wiley.com/en-gb/Greene %27s+Protective+Groups+in+Organic+Synthesis %2C+4th+Edition-p-9780470053485.
Knopman, David S., Helene Amieva, Ronald C. Petersen, Gael Ch6telat, David M. Holtzman, Bradley T. Hyman, Ralph A. Nixon, and David T. Jones. 2021. “Alzheimer Disease.” Nature Reviews. Disease Primers 7 (1): 33. https://doi.org/10.1038/s41572-021-00269-y.
Kraeuter, Ann-Katrin, Paul C. Guest, and Zoltin Sarnyai. 2019. “The Y-Maze for Assessment of Spatial Working and Reference Memory in Mice.” In Pre-Clinical Models, edited by Paul C. Guest, 1916:105-11. Methods in Molecular Biology. New York, NY: Springer New York. https://doi.org/10.1007/978-1-4939-8994-2_10.
Sakono, Masafumi, and Tamotsu Zako. 2010. “Amyloid Oligomers: Formation and Toxicity of Ap Oligomers.” The FEBS Journal 277 (6): 1348-58. https://doi.org/10.1111/j.1742-4658.2010.07568.x.
Thomas Bayer. 2012. “Proteinopathies, a Core Concept for Understanding and Ultimately Treating Degenerative Disorders?Elsevier Enhanced Reader.” 2012. https://doi.org/10.1016/j.euroneuro.2013.03.007.
Yeung, Jason H. Y., Beatriz Calvo-Flores Guzmin, Thulani H. Palpagama, Jayarjun Ethiraj, Ying Zhai, Warren P. Tate, Katie Peppercorn, Henry J. Waldvogel, Richard L. M. Faull, and Andrea Kwakowsky. 2020. “Amyloid-Betal-42 Induced Glutamatergic Receptor and Transporter Expression Changes in the Mouse Hippocampus.” Journal of Neurochemistry 155 (1): 62-80. https://doi.org/10.1111/Jnc.15099.

1. A compound according to Formula I: 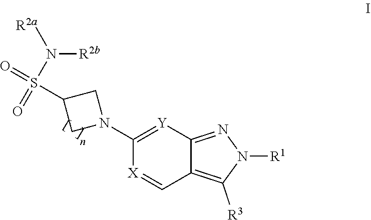
wherein
n is 1, 2 or 3;
one of X and Y is N and the other is CH;
R¹ is phenyl or pyridyl optionally substituted with one or more independently selected R⁴;
R^{2a} and R^{2b} are independently selected from:
H,
C₁₋₄ alkyl optionally substituted with one or more independently selected halo, —CN or C₁₋₄ alkoxy, and
C₃₋₆ cycloalkyl optionally substituted with one or more independently selected halo, —CN or C₁₋₄ alkoxy;
or R^{2a} and R^{2b} together with the N atom may form a 4-7 membered heterocycloalkyl optionally substituted with one or more independently selected halo, —CN or C₁₋₄ alkoxy;
R³ is H, C₁₋₆ alkyl optionally substituted with one or more independently selected halo, or C₃0.7 monocyclic cycloalkyl optionally substituted with one or more independently selected halo; and
each R⁴ is selected from:
OH,
halo,
C₁₋₄ alkyl optionally substituted with one or more independently selected halo or C₁₋₄ alkoxy, or
C₁₋₄ alkoxy optionally substituted with one or more independently selected halo or C₁₋₄ alkoxy;
or a pharmaceutically acceptable salt, or a solvate or the pharmaceutically acceptable salt of a solvate thereof.

2. A compound according to claim 1, or a pharmaceutically acceptable salt, or a solvate or the pharmaceutically acceptable salt of a solvate thereof, wherein n is 0.

3. A compound according to claim 1 or 2, or a pharmaceutically acceptable salt, or a solvate or the pharmaceutically acceptable salt of a solvate thereof, wherein R¹ is phenyl substituted with one or more independently selected R⁴.

4. A compound according to any one of claims 1-2, or a pharmaceutically acceptable salt, or a solvate or the pharmaceutically acceptable salt of a solvate thereof, wherein R¹ is phenyl.

5. A compound according to claim 1 or a pharmaceutically acceptable salt, or a solvate or the pharmaceutically acceptable salt of a solvate thereof, wherein the compound of the invention is according to any of the Formula IIa, IIb, IIc or IId: 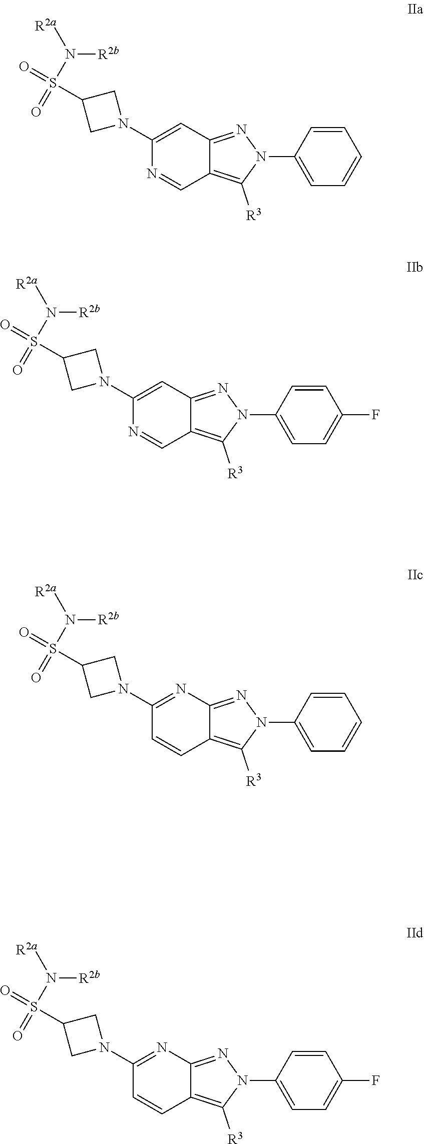
wherein R^{2a}, R^{2b}, and R³ are as described above.

6. A compound according to any one of claims 1-5, or a pharmaceutically acceptable salt, or a solvate or the pharmaceutically acceptable salt of a solvate thereof, wherein R³ is H.

7. A compound according to any one of claims 1-5, or a pharmaceutically acceptable salt, or a solvate or the pharmaceutically acceptable salt of a solvate thereof, wherein R³ is C₁₋₆ alkyl.

8. A compound according to any one of claims 1-5, or a pharmaceutically acceptable salt, or a solvate or the pharmaceutically acceptable salt of a solvate thereof, wherein R³ is C₃₋₇ monocyclic cycloalkyl.

9. A compound according to claim 1 or a pharmaceutically acceptable salt, or a solvate or the pharmaceutically acceptable salt of a solvate thereof, wherein the compound of the invention is according to any of the Formula IIIa, IIIb, IIIc, IIId, IIIe or IIIf: 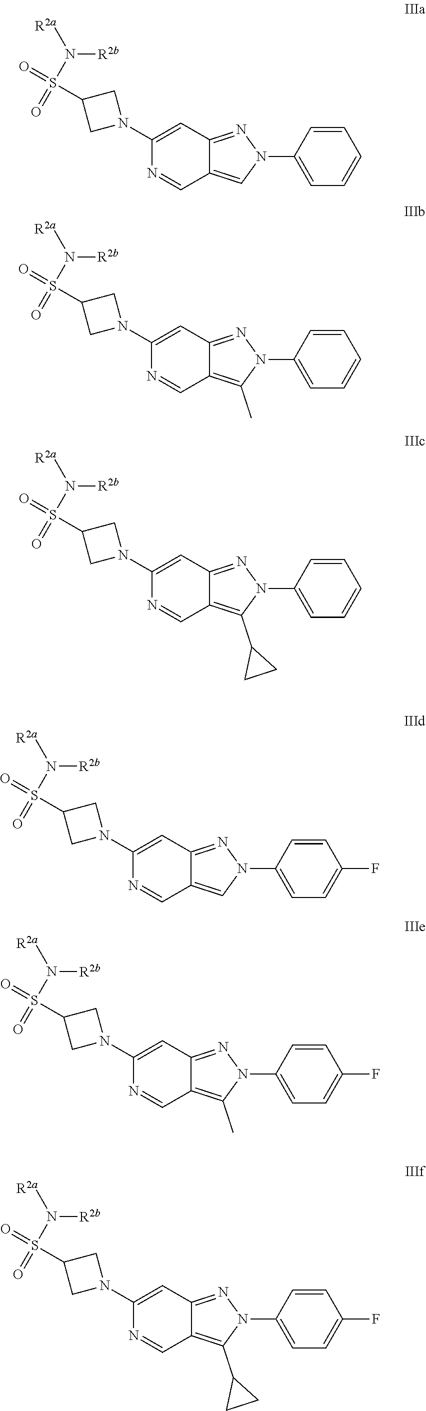
wherein R^{2a} and R^{2b} are as described above.

10. A compound according to any one of claims 1-9, or a pharmaceutically acceptable salt, or a solvate or the pharmaceutically acceptable salt of a solvate thereof, wherein each R^{2a} and R^{2b} are independently selected from H, C₁₋₄ alkyl, and C₃₋₆ cycloalkyl.

11. A compound according to any one of claims 1-9, or a pharmaceutically acceptable salt, or a solvate or the pharmaceutically acceptable salt of a solvate thereof, wherein R^{2a} is C₁₋₄ alkyl and R^{2b} is H, C₁₋₄ alkyl or C₃₋₆ cycloalkyl.

12. A pharmaceutical composition comprising a pharmaceutically acceptable carrier and a pharmaceutically effective amount of a compound or pharmaceutically acceptable salt thereof according to any one of claims 1-11.

13. A compound or pharmaceutically acceptable salt thereof, according to any one of claims 1-11, or a pharmaceutical composition according to claim 12 for use in medicine.

14. A compound or pharmaceutically acceptable salt thereof, according to any one of claims 1-11, or a pharmaceutical composition according to claim 12 for use in the prophylaxis and/or treatment of neurodegenerative diseases.

15. A compound or pharmaceutically acceptable salt thereof, according to any one of claims 1-11, or a pharmaceutical composition according to claim 12 for use in the prophylaxis and/or treatment of neurodegenerative disease, wherein the neurodegenerative disease is selected from Alzheimer's disease and other dementias, brain cancer, degenerative nerve diseases, encephalitis, epilepsy, genetic brain disorders, head and brain malformations, hydrocephalus, stroke, Parkinson's disease, multiple sclerosis and amyotrophic lateral sclerosis.

16. A compound or pharmaceutically acceptable salt thereof, according to any one of claims 1-11, or a pharmaceutical composition according to claim 12 for use in the treatment of Alzheimer's disease.
